(19) United States
(12) Patent Application Publication
(10) Pub. No.: US 2023/0100193 A1
(43) Pub. Date: Mar. 30, 2023

(54) NOVEL PHARMACEUTICAL FORMULATIONS CONTAINING INDIRUBIN AND DERIVATIVES THEREOF AND METHODS OF MAKING AND USING THE SAME

(71) Applicant: Cytodigm, Inc., Natick, MA (US)

(72) Inventors: Bin Wu, Sharon, MA (US); Paul Boucher, Melrose, MA (US)

(21) Appl. No.: 17/858,404

(22) Filed: Jul. 6, 2022

Related U.S. Application Data
(63) Continuation of application No. 16/986,526, filed on Aug. 6, 2020, now abandoned.
Application No. 16/986,526 is a continuation of application No. 16/582,688, filed on Sep. 25, 2019, now abandoned.
Application No. 16/582,688 is a continuation of application No. PCT/US2018/025075, filed on Mar. 29, 2018.
(60) Provisional application No. 62/478,317, filed on Mar. 29, 2017.

(51) Int. Cl. A61K 9/51; A61K 31/404

(52) U.S. Cl. CPC A61K 9/5153; A61K 31/404; A61K 9/5192

(57) ABSTRACT

The invention described herein provides various indirubin compositions for treating diseases.

### REFERENCE TO RELATED APPLICATION

This application is a continuation application of U.S. patent application Ser. No. 16/986,526, filed on Aug. 6, 2020, which is a continuation application of U.S. patent application Ser. No. 16/582,688, filed on Sep. 25, 2019, which is a continuation application of International Patent Application No. PCT/US2018/025075, filed on Mar. 29, 2018, which claims the benefit of U.S. Provisional Application No. 62/478,317, filed on Mar. 29, 2017, the entire contents of each of which is incorporated herein by reference.

This application is also related to International Patent Application Nos. PCT/US2013/046981, filed on Jun. 21, 2013; and PCT/US2014/071409, filed on Dec. 19, 2014, the entire contents of each of which is also incorporated herein by reference.

### BACKGROUND OF THE INVENTION

Indirubin is extracted from the indigo plant. Indirubin is a constituent of a traditional Chinese herbal formula, Dang Gui Long Hui Wan used in the treatment of chronic myelogenous leukemia (CML). It has also been used in Asia as a systemic treatment for psoriasis.

In vitro and animal studies of indirubin have indicated anti-inflammatory, antitumor and neuroprotective effects of indirubin. Recently researchers discovered that indirubin both blocks the migration of glioblastoma cells, preventing their spread to other areas of the brain, and the migration of endothelial cells, preventing them from forming the new blood vessels that the tumor needs to grow. Glioblastomas occur in about 18,500 Americans annually and kill nearly 13,000 of them. Glioblastoma multiforme is the most common and lethal form of the malignancy, with an average survival of 15 months after diagnosis.

Indirubin also inhibits cyclin-dependent kinases in tumor cells. A derivative of indirubin was shown to enhance the cytotoxic effects of Adriamycin. A small clinical study of indirubin in patients with head and neck cancer found a reduction in mucosal damage from radiation therapy. Meisoindigo, a metabolite of indirubin, has also been shown to have similar properties. Positive effects following long term use of indirubin for the treatment of CML have been reported.

The findings suggest that indirubin simultaneously targets tumor invasion and angiogenesis and that drugs of the indirubin family may improve survival in glioblastoma.

However, indirubin has a poor aqueous solubility and poor permeability, which limit its bioavailability, efficacy and delivery. Therefore, there exists a need in the art for indirubin formulations that can increase solubility, bioavailability, improve clinical efficacy, reduce patient dose variation, and potentially reduce side effects.

### SUMMARY OF THE INVENTION

One aspect of the invention provides a pharmaceutical formulation comprising indirubin or an indirubin derivative, and a pharmaceutically acceptable polymer, wherein the pharmaceutically acceptable polymer encapsulates the indirubin or indirubin derivative to form particulates.

In certain embodiments, the average particle size of the particulates is about 1 nm to about 1,000 nm, about 10 nm to about 300 nm, about 20-500 nm, about 20 nm to about 200 nm, about 50-100 nm; or about 100 nm.

In certain embodiments, solubility in an aqueous solution of the indirubin or indirubin derivative in the pharmaceutical formulation is at least about 100%, 2-fold, 3-fold, 5-fold, 10-fold, 20-fold, 50-fold, or 100-fold of that the indirubin or indirubin derivative in the same aqueous solution.

In certain embodiments, the pharmaceutically acceptable polymer is selected from the group consisting of: PLA, PLGA, PEG-PLGA copolymer, PEG-PLA copolymer, PEG-PGA copolymer, poly(ethylene glycol), polycaprolactone, polyanhydrides, poly(ortho esters), polycyanoacrylates, poly(hydroxyalkanoate)s, poly(sebasic acid), polyphosphazenes, polyphosphoesters, modified poly(saccharide)s, and mixtures and copolymers thereof.

In certain embodiments, the pharmaceutically acceptable polymer is PLGA, or a copolymer of PLGA (e.g., PEG-PLGA).

In certain embodiments, the pharmaceutically acceptable polymer comprises a functional group selected from the group containing of: carboxyl, amino, diamine, thiol, aldehyde, hydroxysuccinimide ester, dihydrazide, hydroxysuccinimide-sulfonic acid, maleimide, and azide.

In certain embodiments, the particulates have an incorporated color dye or fluorescent dye.

In certain embodiments, the indirubin derivative is 6-bromoindirubin-3′-oxime (6-BIA).

Another aspect of the invention provides a method of producing a pharmaceutical formulation comprising indirubin or an indirubin derivative, and a pharmaceutically acceptable polymer, wherein the pharmaceutically acceptable polymer encapsulates the indirubin or indirubin derivative to form particulates, the method being a single emulsion process comprising: (a) dissolving indirubin or an indirubin derivative along with a pharmaceutically acceptable polymer in a first solvent to form a polymer-indirubin solution; (b) emulsifying the polymer-indirubin solution in a second solvent to form an emulsion, wherein the first solvent is not miscible or only partially miscible with the second solvent; and (c) removing the first solvent to form the particulates.

In certain embodiments, the average particle size of the particulates is about 1 nm to about 1,000 nm, about 10 nm to about 300 nm, about 20-500 nm, about 20 nm to about 200 nm, about 50-100 nm; or about 100 nm.

In certain embodiments, in step (a), the indirubin or derivative thereof is dissolved in a first portion of the first solvent to form an indirubin solution, before being mixed with a separately prepared polymer solution in a second portion of the first solvent.

In certain embodiments, the polymer-indirubin solution further comprises a surfactant.

In certain embodiments, a surfactant is dissolved in the second solvent before step (b).

In certain embodiments, the method further comprises dissolving or dispersing an additional API in the second solvent before forming the emulsion.

In certain embodiments, the method further comprises dissolving or dispersing a first additional API (other than indirubin or its derivative) in the first solvent and dissolving or dispersing a second additional API (other than indirubin or its derivative) in the second solvent.

In certain embodiments, emulsification is performed using a method selected from the group consisting of: sonication, stirring, homogenization, microfluidization and combination thereof.

In certain embodiments, the method further comprises adsorbing or conjugating a biologic or a chemical entity to the surface of said indirubin particle.

In certain embodiments, the indirubin derivative is 6-bromoindirubin-3′-oxime (6-BIA).

Another aspect of the invention provides a method of producing a pharmaceutical formulation comprising indirubin or an indirubin derivative, and a pharmaceutically acceptable polymer, wherein the pharmaceutically acceptable polymer encapsulates the indirubin or indirubin derivative to form particulates, the method being a double emulsion process comprising: (a) dissolving indirubin or an indirubin derivative along with a pharmaceutically acceptable polymer in a first solvent to form a polymer-indirubin solution; (b) adding a small amount (e.g., 0.5% (v/v), 1% (v/v), 5% (v/v)) of a second solvent to the polymer-indirubin solution to form a mixture, wherein the first solvent is not miscible or only partially miscible with the second solvent; (c) emulsifying the mixture to form a first emulsion; (d) emulsifying the first emulsion in a third solvent to form a second emulsion; and, (e) removing the first solvent to form said particulates.

In certain embodiments, the average particle size of the particulates is about 1 nm to about 1,000 nm, about 10 nm to about 300 nm, about 20-500 nm, about 20 nm to about 200 nm, about 50-100 nm; or about 100 nm.

In certain embodiments, the second and the third solvents are the same solvent.

In certain embodiments, the second and the third solvents are both water.

In certain embodiments, the third solvent further comprises a surfactant.

In certain embodiments, the surfactant is selected from the group consisting of: detergents, wetting agents, emulsifiers, foaming agents, and dispersants.

In certain embodiments, the surfactant is polyvinyl alcohol (PVA).

In certain embodiments, the method further comprises dissolving or dispersing an additional API in the second solvent before forming the first emulsion.

In certain embodiments, the method further comprises dissolving or dispersing a first additional API (other than indirubin or its derivative) in the first solvent and dissolving or dispersing a second additional API (other than indirubin or its derivative) in the second solvent.

In certain embodiments, emulsification is performed using a method selected from the group consisting of: sonication, stirring, homogenization, microfluidization and combination thereof.

In certain embodiments, the method further comprises adsorbing or conjugating a biologic or a chemical entity to the surface of said indirubin particle.

In certain embodiments, the first solvent is not miscible with water, or is selected from the group consisting of. ethyl acetate, dichloromethane, and chloroform.

In certain embodiments, a water-miscible solvent is mixed with a non-water-miscible solvent as a co-solvent for the dissolution of the polymer or the APIs or both.

In certain embodiments, the second solvent is water, or wherein the third solvent is water.

In certain embodiments, the polymer solution has a concentration selected from the group consisting of: 1 μg/mL-1 g/mL (w/w), 1 mg/mL-500 mg/mL (w/w), and 10 mg/mL-100 mg/mL (w/w).

In certain embodiments, the indirubin derivative is 6-bromoindirubin-3′-oxime (6-BIA).

Another aspect of the invention provides a method of producing a pharmaceutical formulation comprising indirubin or an indirubin derivative, and a pharmaceutically acceptable polymer, wherein the pharmaceutically acceptable polymer encapsulates the indirubin or indirubin derivative to form particulates, the method being a precipitation process comprising: (1) dissolving indirubin or a derivative thereof in a first solvent along with a pharmaceutically acceptable polymer; (2) optionally adding to the first solvent a first solution comprising a surface stabilizer to form a formulation; and, (3) precipitating the formulation from step (2) into a second solution containing the surface stabilizer in a second solvent, wherein the second solvent is miscible with the first solvent and is a non-solvent for both the polymer and the indirubin or the derivative thereof.

In certain embodiments, the first solvent is selected from the group consisting of: DMSO, DMF, acetone, alcohols, acetonitrile, and THF.

In certain embodiments, the second solvent is selected from the groups consisting of: water, methanol, ethanol, isopropyl alcohol, benzyl alcohol. In certain embodiments, the second solvent is water.

In certain embodiments, the method further comprises removing unwanted stabilizer or any impurity, if present, by dialysis or diafiltration.

In certain embodiments, the average particle size of the particulates is about 1 nm to about 1,000 nm, about 10 nm to about 300 nm, about 20-500 nm, about 20 nm to about 200 nm, about 50-100 nm; or about 100 nm.

In certain embodiments, the indirubin derivative is 6-bromoindirubin-3′-oxime (6-BIA).

Another aspect of the invention provides a method of treating cancer in a subject in need thereof comprising administering an effective amount of the subject pharmaceutical composition.

In certain embodiments, the cancer is glioblastoma or leukemia.

In certain embodiments, the subject is a human.

Another aspect of the invention provides a method of treating an inflammatory disease in a subject in need thereof comprising administering an effective amount of the subject pharmaceutical composition.

In certain embodiments, the inflammatory disease is psoriasis.

In certain embodiments, the subject is a human.

Another aspect of the invention provides a method of treating a neurodegenerative disorder in a subject in need thereof comprising administering an effective amount of the subject pharmaceutical composition.

In certain embodiments, the neurodegenerative disorder is Alzheimer's disease.

In certain embodiments, the subject is a human.

Another aspect of the invention provides a method of treating a disorder associated with abnormal GSK-3 activity, in a subject in need thereof, the method comprising administering an effective amount of the subject pharmaceutical composition.

In certain embodiments, the disorder is Type II diabetes (Diabetes mellitus type 2), Alzheimer's Disease, inflammation, cancer (e.g., glioma and pancreatic cancer), or bipolar disorder.

In certain embodiments, the subject is a human.

It should be understood that any one embodiment described herein, including those described only in the Examples or only under one section of the Detailed Description, can be combined with any one or more other embodiments unless expressly disclaimed or improper.

### DETAILED DESCRIPTION OF THE INVENTION

The present disclosure provides novel pharmaceutical formulations containing indirubin or derivatives thereof (hereinafter collectively “indirubin” for simplicity) for the treatment of various human diseases.

The pharmaceutical formulation of the invention is partly based on the surprising discovery that solubility and bioavailability of indirubin can be improved by encapsulating indirubin particles in nanoparticles of certain polymers, such as biodegradable and biocompatible polymers PLA or PLGA. Encapsulation can be achieved using any of the methods described herein.

Polymers, especially biodegradable and biocompatible polymers, have been widely used to encapsulate active pharmaceutical ingredients (APIs) into microspheres and nanoparticles. For example, microspheres based on polylactide, PLA, and poly(lactide-co-glycolide), PLGA, are the basis for numerous commercial depot products such as Lupron Depot and Bydureon. These microspheres, however, have been used mainly to offer sustained drug release (e.g., for weeks or months of sustained drug release). In addition, PLGA, PLA and other biodegradable polymers have also been used to encapsulate drugs into nanoparticles for targeted drug delivery. Applicant is not aware of the use of such microspheres and nanoparticles to intentionally increase solubility/bioavailability of poorly water-soluble APIs, or whether such microspheres and nanoparticles can encapsulate poorly water-soluble APIs or can be used to increase solubility/bioavailability of poorly water-soluble APIs at all, especially an extremely insoluble compound like indirubin.

It is surprisingly discovered that indirubin and its derivatives can be encapsulated into nanoparticles of biodegradable polymers, such as PLGA and PLA. More importantly, nanoparticles of biodegradable polymers, such as PLGA and PLA, can be used to dramatically increase the dissolution rate of indirubin and its derivatives by encapsulating them into nanoparticles of such biodegradable polymers.

Furthermore, it is surprisingly discovered that indirubin and its derivatives can be encapsulated into nanoparticles of biodegradable polymers, such as PLGA and PLA, substantially without surface stabilizers that are adsorbed on or associated with the surface of indirubin or derivatives thereof. Such surface stabilizers include anionic surface stabilizers, cationic surface stabilizers, zwitterionic surface stabilizers, and ionic surface stabilizers, which are described in, for example, WO2013/192493 (incorporated herein by reference). That is, to the extent that any surface stabilizers are present at all in the nanoparticles of biodegradable polymers encapsulating indirubin, the surface stabilizers are not in direct contact with the surface of indirubin or derivatives thereof.

Therefore, the instant invention provides a pharmaceutical formulation comprising indirubin or an indirubin derivative, and at least one pharmaceutically acceptable polymer, wherein the pharmaceutically acceptable polymer encapsulates the indirubin or indirubin derivative to form particulates.

In certain embodiments, the particulates are substantially devoid of surface stabilizers that are adsorbed on or associated with the surface of indirubin or derivatives thereof.

In certain embodiments, the particles are microparticles or nanoparticles. For example, the particles may be nanoparticles. Optionally, the nanoparticles have average particle sizes of about 1 nm to 500 μm, about 1 nm to 200 μm, about 1 nm to 100 μm, about 1 nm to 50 μm, about 1 nm to 10 μm, about 1 nm to 5 μm, about 1 nm to about 1,000 nm, about 10 nm to about 300 nm, about 20-500 nm, about 20 nm to about 200 nm, about 50-100 nm; or about 100 nm.

In certain embodiments, solubility in an aqueous solution (e.g., water) of said indirubin or indirubin derivative in said pharmaceutical formulation is at least about 100%, 2-fold, 3-fold, 5-fold, 10-fold, 20-fold, 50-fold, or 100-fold of that said indirubin or indirubin derivative in the same aqueous solution.

In certain embodiments, the pharmaceutically acceptable polymer is selected from the group consisting of: PLA, PLGA, PEG-PLGA copolymer, PEG-PLA copolymer, PEG-PGA copolymer, poly(ethylene glycol), polycaprolactone, polyanhydrides, poly(ortho esters), polycyanoacrylates, poly(hydroxyalkanoate)s, poly(sebasic acid), polyphosphazenes, polyphosphoesters, modified poly(saccharide)s, mixtures and copolymers thereof.

In certain embodiments, the pharmaceutically acceptable polymer is PLGA, or a copolymer of PLGA (e.g., PEG-PLGA).

In certain embodiments, the pharmaceutically acceptable polymer optionally comprises a functional group. For example, the functional group may be selected from the group containing of: carboxyl, amino, diamine, thiol, aldehyde, hydroxysuccinimide ester, dihydrazide, hydroxysuccinimide-sulfonic acid, maleimide, and azide.

In certain embodiments, a color dye or fluorescent dye can be incorporated into the nanoparticles to facilitate the imaging of the particles.

This invention also provides a method for making the subject pharmaceutical composition/formulation (of polymeric particles) comprising indirubin or its derivatives. More specifically, the invention described herein provides a method for preparing the subject pharmaceutical composition/formulation (of polymeric particles), e.g., a pharmaceutical formulation comprising indirubin or an indirubin derivative, and a pharmaceutically acceptable polymer, wherein the pharmaceutically acceptable polymer encapsulates the indirubin or indirubin derivative to form particulates, the method being a single emulsion process comprising: (a) dissolving indirubin or an indirubin derivative along with a pharmaceutically acceptable polymer in a first solvent to form a polymer-indirubin solution; (b) emulsifying the polymer-indirubin solution in a second solvent to form an emulsion, wherein the first solvent is not miscible or only partially miscible with the second solvent; and (c) removing the first solvent to form the particulates.

In certain embodiments, the particulates are substantially devoid of surface stabilizers that are adsorbed on or associated with the surface of indirubin or derivatives thereof.

In certain embodiments, the particles are microparticles or nanoparticles. For example, the particles may be nanoparticles. Optionally, the nanoparticles have average particle sizes of about 1 nm to 500 μm, about 1 nm to 200 μm, about 1 nm to 100 μm, about 1 nm to 50 μm, about 1 nm to 10 μm, about 1 nm to 5 μm, about 1 nm to about 1,000 nm, about 10 nm to about 300 nm, about 20-500 nm, about 20 nm to about 200 nm, about 50-100 nm; or about 100 nm.

In certain embodiments, the pharmaceutically acceptable polymer is selected from the group consisting of: PLA, PLGA, PEG-PLGA copolymer, PEG-PLA copolymer, PEG-PGA copolymer, poly(ethylene glycol), polycaprolactone, polyanhydrides, poly(ortho esters), polycyanoacrylates, poly(hydroxyalkanoate)s, poly(sebasic acid), polyphosphazenes, polyphosphoesters, modified poly(saccharide)s, mixtures and copolymers thereof.

In certain embodiments, the pharmaceutically acceptable polymer is PLGA, or a copolymer of PLGA (e.g., PEG-PLGA).

In certain embodiments, the pharmaceutically acceptable polymer optionally comprises a functional group. For example, the functional group may be selected from the group containing of: carboxyl, amino, diamine, thiol, aldehyde, hydroxysuccinimide ester, dihydrazide, hydroxysuccinimide-sulfonic acid, maleimide, and azide.

In certain embodiments, a color dye or fluorescent dye can be incorporated into the nanoparticles to facilitate the imaging of the particles.

In certain embodiments, in step (a) (before the emulsification step (b)), the indirubin or derivative thereof is dissolved in a first portion of the first solvent to form an indirubin solution, before being mixed with a separately prepared polymer solution in a second portion of the first solvent.

In certain embodiments, the polymer-indirubin solution further comprises a surfactant.

In certain embodiments, a surfactant is optionally dissolved in the second solvent before step (b) (emulsification).

In certain embodiments, the method further comprises dissolving or dispersing an additional API in the second solvent before forming the emulsion. In certain embodiments, the API is soluble in the second solvent. In certain embodiments, the API is a biologic entity. For example, the biologic entity may be selected from the group consisting of a protein, a peptide, a growth factor, an oligonucleotide, an antibody, a polycarbohydrate, an enzyme, an amino acid, a DNA, an RNA, and a ligand. In certain embodiments, the API is effective to treat a disease or condition treatable by indirubin or derivative thereof.

In certain embodiments, the API is selected from: amino acids, proteins, peptides, nucleotides, anti-obesity drugs, nutraceuticals, dietary supplements, central nervous symptom stimulants, carotenoids, corticosteroids, elastase inhibitors, anti-fungals, alkylxanthine, oncology therapies, anti-emetics, analgesics, opioids, antipyretics, cardiovascular agents, anti-inflammatory agents, anthelmintics, antianhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, alpha-adrenergic receptor blocking agents, beta-adrenoceptor blocking agents, blood products, blood substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants, anoretics, sympathomimetics, thyroid agents, vasodilators, vasomodulator, xanthines, Mu receptor antagonists, Kappa receptor antagonists, non-narcotic analgesics, monoamine uptake inhibitors, adenosine regulating agents, cannabinoids, Substance P antagonists, neurokinin-1 receptor antagonists, and sodium channel blockers. The nutraceutical can be selected from lutein, folic acid, fatty acids, fruit extracts, vegetable extracts, vitamin supplements, mineral supplements, phosphatidylserine, lipoic acid, melatonin, glucosanline/chondroitin, Aloe Vera, Guggul, glutamine, amino acids, green tea, lycopene, whole foods, food additives, herbs, phytonutrients, antioxidants, flavonoid constituents of fruits, evening primrose oil, flax seeds, fish oils, marine animal oils, and probiotics.

In certain embodiments, the method further comprises dissolving or dispersing a first additional API (other than indirubin or its derivative) in the first solvent and dissolving or dispersing a second additional API (other than indirubin or its derivative) in the second solvent. In certain embodiments, the first additional API is soluble in the first solvent. In certain embodiments, the second additional API is soluble in the second solvent. In certain embodiments, the first additional API is a biologic entity. In certain embodiments, the second additional API is a biologic entity. In certain embodiments, the first and/or the second API is effective to treat a disease or condition treatable by indirubin or derivative thereof.

In certain embodiments, emulsification is performed using a method selected from the group consisting of: sonication, stirring, homogenization, microfluidization and combination thereof. In one embodiment, the emulsification is performed using microfluidization. In certain embodiments, the microfluidization is performed at an applied pressure selected from the group consisting of 1-100,000 psi, 1,000-70,000 psi, and 5,000-30,000 psi. In certain embodiments, the microfluidization is performed at a flow rate of 1 mL/min-100 L/min, preferably 1 mL/min-1 L/min. In certain embodiments, the emulsion is cycled through the microfluidizer 1-100 times, preferably 2-10 times.

The method described above may further includes, after the first emulsification step (b), a step of adding a third solvent and emulsifying again in the presence of the third solvent in order to form a second emulsion, but before removing the first solvent.

Thus in a related aspect, the invention also provides a method for preparing polymeric particles, e.g., a method of producing a pharmaceutical formulation comprising indirubin or an indirubin derivative, and a pharmaceutically acceptable polymer, wherein the pharmaceutically acceptable polymer encapsulates the indirubin or indirubin derivative to form particulates, the method being a double emulsion process comprising: (a) dissolving indirubin or an indirubin derivative along with a pharmaceutically acceptable polymer in a first solvent to form a polymer-indirubin solution; (b) adding a small amount (e.g., 0.5% (v/v), 1% (v/v), 5% (v/v)) of a second solvent to the polymer-indirubin solution to form a mixture, wherein the first solvent is not miscible or only partially miscible with the second solvent; (c) emulsifying the mixture to form a first emulsion; (d) emulsifying the first emulsion in a third solvent to form a second emulsion; and, (e) removing the first solvent to form said particulates.

In certain embodiments, the particulates are substantially devoid of surface stabilizers that are adsorbed on or associated with the surface of indirubin or derivatives thereof.

In certain embodiments, the particles are microparticles or nanoparticles. For example, the particles may be nanoparticles. Optionally, the nanoparticles have average particle sizes of about 1 nm to 500 μm, about 1 nm to 200 μm, about 1 nm to 100 μm, about 1 nm to 50 μm, about 1 nm to 10 μm, about 1 nm to 5 μm, about 1 nm to about 1,000 nm, about 10 nm to about 300 nm, about 20-500 nm, about 20 nm to about 200 nm, about 50-100 nm; or about 100 nm.

In certain embodiments, the second and third solvents are the same solvent, and optionally, the same solvent is water.

In certain embodiments, the third solvent further comprises a surfactant. Optionally, the surfactant is selected from the group consisting of detergents, wetting agents, emulsifiers, foaming agents, and dispersants. Optionally, the surfactant is polyvinyl alcohol.

In certain embodiments, the method further comprises dissolving or dispersing an additional API in the second solvent before emulsification. In certain embodiments, the API is soluble in the second solvent. In certain embodiments, the API is a biologic entity. For example, the biologic entity may be selected from the group consisting of a protein, a peptide, a growth factor, an oligonucleotide, an antibody, a polycarbohydrate, an enzyme, an amino acid, a DNA, an RNA, and a ligand. In certain embodiments, the API is effective to treat a disease or condition treatable by indirubin or derivative thereof.

In certain embodiments, the method further comprises dissolving or dispersing a first additional API (other than indirubin or its derivative) in the first solvent and dissolving or dispersing a second additional API (other than indirubin or its derivative) in the second solvent. In certain embodiments, the first additional API is soluble in the first solvent. In certain embodiments, the second additional API is soluble in the second solvent. In certain embodiments, the first additional API is a biologic entity. In certain embodiments, the second additional API is a biologic entity. In certain embodiments, the first and/or the second API is effective to treat a disease or condition treatable by indirubin or derivative thereof.

In certain embodiments, emulsification is performed using a method selected from the group consisting of: sonication, stirring, homogenization, microfluidization and combination thereof. In one embodiment, the emulsification is performed using microfluidization. In certain embodiments, the microfluidization is performed at an applied pressure selected from the group consisting of 1-100,000 psi, 1,000-70,000 psi, and 5,000-30,000 psi. In certain embodiments, the microfluidization is performed at a flow rate of 1 mL/min-100 L/min, preferably 1 mL/min-1 L/min. In certain embodiments, the emulsion is cycled through the microfluidizer 1-100 times, preferably 2-10 times.

In certain embodiments, the method further comprises adsorbing or conjugating biologic or chemical entities to the surface of said indirubin particles.

In another embodiment, the first solvent is not miscible with water. For example, the first solvent may be selected from the group containing ethyl acetate, dichloromethane, and chloroform. Optionally a water-miscible solvent can be mixed with the non water-miscible solvent as a co-solvent for the dissolution of the polymer or the APIs or both.

In another embodiment, the second solvent is ethanol or water. In another embodiment, the second solvent is water.

In another embodiment, the third solvent is ethanol or water. In another embodiment, the third solvent is water.

In another embodiment, the polymer solution has a concentration selected from the group consisting of 1 μg/mL-1 g/mL percent by weight, 1 mg/mL-500 mg/mL percent by weight, and 10 mg/mL-100 mg/mL percent by weight.

A related aspect of the invention provides a method for preparing polymeric particles, e.g., a method of producing a pharmaceutical formulation comprising indirubin or an indirubin derivative, and a pharmaceutically acceptable polymer, wherein the pharmaceutically acceptable polymer encapsulates the indirubin or indirubin derivative to form particulates, the method being a microprecipitation process comprising: (1) dissolving indirubin or a derivative thereof in a first solvent along with a pharmaceutically acceptable polymer; (2) optionally adding to the first solvent a first solution comprising a surface stabilizer to form a formulation; and, (3) precipitating the formulation from step (2) into a second solution containing the surface stabilizer in a second solvent, wherein the second solvent is miscible with the first solvent and is a non-solvent for both the polymer and the indirubin or the derivative thereof.

In certain embodiments, the first solvent is selected from the group consisting of: DMSO, DMF, acetone, alcohols, acetonitrile, and THF.

In certain embodiments, the second solvent is selected from the groups consisting of: water, methanol, ethanol, isopropyl alcohol, benzyl alcohol. In certain embodiments, the second solvent is water.

In certain embodiments, the method further comprises removing unwanted stabilizer or any impurity, if present, by dialysis or diafiltration. Optionally, the method further comprises concentrating the dispersion by any conventional means.

In certain embodiments, the average particle size of the particulates is about 1 nm to about 1,000 nm, about 10 nm to about 300 nm, about 20-500 nm, about 20 nm to about 200 nm, about 50-100 nm; or about 100 nm.

It should be understood that one of skill in the art can readily combine any one embodiment described herein, including the specific examples below, with any other embodiment(s) of the invention within the spirits of the invention.

### Indirubin and Derivatives Thereof

The methods of the invention can be used to encapsulate indirubin or its derivatives, analogs, salts, solvates, congeners, bioisosteres, hydrolysis products, metabolites, precursors, and prodrugs thereof.

The molecular structure of indirubin is shown below.

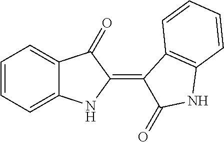 [SMILES: O=C1Nc2ccccc2/C1=C1/Nc2ccccc2C1=O]

In certain embodiments, derivatives of indirubin may include mesoindigo, indirubin 3′ oximes (e.g., indirubin-3′-oxime, 5′-nitro-indirubinoxime, 5′-fluoro-indirubinoxime, 5′-bromo-indirubin-3′-monoxime, 6′-bromo-indirubin-3′-monoxime, 7′-bromo-indirubin-3′-monoxime, and 5′-trimethylacetamino-indirubinoxime), IDR-E804 (Shim et al., BMC Cancer, 12:164 (May 3, 2012), indirubin hydrazone derivatives, or pharmaceutically or physiologically acceptable salt thereof.

In certain embodiments, derivatives of indirubin may include 5-iodo-indirubin-3′-monoxime, 5-bromo-indirubin, 5-chloro-indirubin, 5-fluoro-indirubin, 5-methyl-indirubin, 5-nitro-indirubin, 5-503H-indirubin, 5′-bromo-indirubin, 5-5′-dibromo-indirubin, 5′-bromo-indirubin 5-sulfonic acid, indirubin-5-sulfonic acid sodium salt, 5-5′-dibromo-indirubin 5-sulfonic acid-indirubin-3′-oxime, indirubin-3′-acetoxime, indirubin-3′-methoxime, N-acetyl-indirubin, 5-NH-trimethylacetyl-indirubin-3-oxime, indirubin-5-nitro-3′-oxime (INO), 5-halogeno-indirubin, N-ethyl-indirubin, N-methylisoindigo, 6-hydroxy-5-methylindirubin, 6,7′-dihydroxy-5-methylindirubin, or indirubin-3′-(2,3 dihydroxypropyl)-oximether, or pharmaceutically or physiologically acceptable salt thereof.

In certain embodiments, derivatives of indirubin may include: (1) indirubin 3′-monooxime; (2) indirubin 5-sulfonic acid; (4) 1H,1′H-[2,3] biindolylidene-3,2′-dione; (5) 5-fluoro-1H,1′H-[2,3] biindolylidene-3,2′-dione; (6) 1H,1′H-[2,3] biindolylidene-3,2′-dione-3-oxime; (7) 1-acetyl-1H,1′H-[2,3] biindolylidene-3,2′-dione; (8) 5′-nitro-1H,1′H-[2,3] biindolylidene-3,2′-dione; (9) 5′-nitro-1H,1′H-[2,3] biindolylidene-3,2′-dione-3-oxime; (10) 5-fluoro-1H,1′H-[2,3] biindolylidene-3,2′-dione-3-oxime; (11) 5′-methyl-1H,1′H-[2,3] biindolylidene-3,2′-dione-3-oxime; (12) 5′-chloro-1H,1′H-[2,3] biindolylidene-3,2′-dione-3-oxime; (13) 5′-iodo-1H,1′H-[2,3] biindolylidene-3,2′-dione-3-oxime; (14) 5′,7′-dimethyl-1H,1′H-[2,3] biindolylidene-3,2′-dione-3-oxime; (15) 5′-chloro-7′-methyl-1H,1′H-[2,3] biindolylidene-3,2′-dione-3-oxime; (16) 5-bromo-1H,1′H-[2,3] biindolylidene-3,2′-dione-3-oxime; (17) 3,2′-dioxo-1,3,1′,2′-tetrahydro-[2,3′]biindolylidene-5′-sodium sulfonate; (18) 3-hydroxyimino-2′-oxo-,3,1′,2′-tetrahydro-[2,3′]biindolylidene-5′-sodium sulfonate; (19) 5-bromo-1H,1′H-[2,3]-biindolylidene-3,2′-dione; (20) 5-bromo-5′-nitro-1H,1′H-[2,3′]-biindolylidene-3,2′-dione-3-oxime; (21) 5′-methyl-1H,1′H-[2,3]-biindolylidene-3,2′-dione; (22) 5′-chloro-1H,1′H-[2,3]-biindolylidene-3,2′-dione; (23) 5′-iodo-1H,1′H-[2,3′]-biindolylidene-3,2′-dione; (24) 5′,7′-dimethyl-1H,1′H-[2,3]-biindolylidene-3,2′-dione; (25) 5′-chloro,7′-methyl-1H,1′H-[2,3′]-biindolylidene-3,2′-dione; (26) 5′-amino-1H,1′H-[2,3′]-biindolylidene-3,2′-dione; (27) 5-NH-trimethylacetyl-indirubin-3-oxime; (28) 5′-amino-1H,1′H-[2,3]-biindolylidene-3,2′-dione-3-oxime; (29) 6-hydroxy-5-methylindirubin; (30) 6,7′-dihydroxy-5-methylindirubin; (31) 3,4,5-trihydroxy-6-(5-methyl-1H,1′H-[2′,3]bis-indolyliden-2,3′-dion-6-yl)-tetrahydropyran-2-carboxylic acid; (32) 3,4,5-trihydroxy-6-(7′-hydroxy-5-methyl-1H1′H-[2′,3]bisindolyliden-2,3′-dion-6-yl)-tetrahydropyran-2-carboxylic acid; (33) 5-methylindirubin; (34) indirubin-5-sulfonamide; (35) indirubin-5-sulfonic acid (2-hydroxyethyl)-amide; (36) 5-iodoindirubin-3′-monooxime; (37) 5-fluoroindirubin; (38) 5,5′-dibromoindirubin; (39) 5-nitroindirubin; (40) 5-bromoindirubin; (41) (2′Z,3′E)-6-bromoindirubin-3′-oxime (B 10); (42) 5-iodoindirubin; (43) indirubin-5-sulfonic acid-3′-monooxime; (44) 3,4-bis(1-methylindole-3-yl)-1H-pyrrole-2,5-dione; (45) 3-[1-methylindole-3-yl)-4-(1-propylindole-3-yl)-1H-pyrrole-2,5-dione; (46) 3-[1-(3-cyanopropyl)indole-3-yl]-4-(1-methyl-indole-3-yl)-1H-pyrrole-2,5-dione; (47) 3-[1-(3-aminopropyl)-indole-3-yl]-4-(1-methylindole-3-yl)-1H-pyrrole-2,5-dione; (48) 3-[1-(3-carboxypropyl)indole-3-yl]-4-(1-methyl-indole-3-yl)-1H-pyrrole-2,5-dione; (49) 3-[1-(3-carbamoyl-propyl)indole-3-yl]-4-(1-methylindole-3-yl)-1H-pyrrole-2,5-dione; (50) 3-[1-(3-aminopropyl)indole-3-yl]-4-(1-methyl-5-propyloxyindole-3-yl)-1H-pyrrole-2,5-dione; (51) 3-[1-(3-hydroxypropyl)indole-3-yl]-4-(1-methyl-5-phenylindole-3-yl)-1H-pyrrole-2,5-dione; (52) 3-[1-(3-aminopropyl)indole-3-yl]-4-(1-methyl-5-phenylindole-3-yl)-1H-pyrrole-2,5-dione; (53) 3-[1-(3-hydroxypropyl)indole-3-yl]-4-(1-methyl-5-methoxycarbonylindole-3-yl)-1H-pyrrole-2,5-dione; (54) 3-[1-(3-hydroxypropyl)indole-3-yl]-4-(1-methyl-5-nitroindole-3-yl)-1H-pyrrole-2,5-dione; (55) 3-(1-methylindole-3-yl)-4-[1-(3-hydroxypropyl)-5-nitroindole-3-yl]-1H-pyrrole-2,5-dione; (56) 3-(2-chlorophenyl)-4-(1-methylindole-3-yl)-1H-pyrrole-2,5-dione; (57) 3-(2,4-dichlorophenyl)-4-(1-methylindole-3-yl)-1H-pyrrole-2,5-dione; (58) 3-(2-chlorophenyl)-4-[1-(3-hydroxypropyl)indole-3-yl]-1H-pyrrole-2,5-dione; (59) 4-[1-(3-aminopropyl)indole-3-yl]-3-(2-chlorophenyl)-1H-pyrrole-2,5-dione; (60) 7,12-dihydro-indolo[3,2-d][1]benzazepin-6(5H)-one; (61) 2-bromo-7,12-dihydro-indolo[3,2-d][1]benzazepin-6(5H)-one; (62) 9-bromo-7,12-dihydro-indolo[3,2-d][1]benzazepin-6(5H)-one; (63) 9-chloro-7,12-dihydro-indolo[3,2-d][1]benzazepin-6(5H)-one; (64) 11-chloro-7,12-dihydro-indolo[3,2-d][1]benzazepin-6(5H)-one; (65) 10-bromo-7,12-dihydro-indolo[3,2-d][1]benzazepin-6(5H)-one; (66) 8-bromo-6,11-dihydro-thieno[3′,2′:2,3]azepino-[4,5-b]indol-5(4H)-one; (67) 9-bromo-7,12-dihydro-4-methoxy-indolo[3,2-d][1]benzazepin-6(5H)-one; (68) 9-bromo-7,12-dihydro-4-hydroxy-indolo[3,2-d][1]benzazepin-6(5H)-one; (69) 7,12-dihydro-4-methoxy-indolo[3,2-d][1]benzazepin-6(5H)-one; (70) 9-bromo-7,12-dihydro-2,3-dimethoxy-indolo[3,2-d][1]benzazepin-6(5H)-one; (71) 9-bromo-7,12-dihydro-2,3-di-hydroxy-indolo[3,2-d][1]benzazepin-6(5H)-one; (72) 7,12-dihydro-2,3-dimethoxy-indolo[3,2-d][1]benzazepin-6(5H)-one; (73) 7,12-dihydro-9-trifluormethyl-indolo[3,2-d][1]benzazepin-6(5H)-one; (74) 7,12-dihydro-2,3-dimethoxy-9-trifluoromethyl-indolo[3,2-d][1]benzazepin-6-(5H)-one; (75) 2-bromo-7,12-dihydro-9-trifluoromethyl-indolo[3,2-d][1]benzazepin-6(5H)-one; (76) 9-bromo-7,12-dihydro-indolo[3,2-d][1]benzazepin-6(5H)-thione; (77) 9-bromo-5,12-bis-(t-butyloxycarbonyl)-7,12-dihydro-indolo[3,2-d][1]benzazepin-6(5H)-one; (78) 9-bromo-12-(t-butyloxycarbonyl)-7,12-dihydro-indolo[3,2-d][1]benzazepin-6-(5H)-one; (79) 9-bromo-5,7-bis-(t-butyloxycarbonyl)-7,12-dihydro-indolo[3,2-d][1]benzazepin-6(5H)-one; (80) 9-bromo-5,7,12-tri-(t-butyloxycarbonyl)-7,12-dihydro-indolo[3,2-d][1]benzazepin-6(5H)-one; (81) 9-bromo-7,12-dihydro-5-methyloxycarbonylmethyl-indolo[3,2-d][1]benzazepin-6(5H)-one; (82) 9-bromo-7,12-dihydro-12-methyloxycarbonylmethyl-indolo[3,2-d][1]benzazepin-6(5H)-one; (83) 9-bromo-7,12-dihydro-12-(2-hydroxyethyl)-indolo[3,2-d][1]benzazepin-6-(5H)-one; (84) 2,9-dibromo-7,12-dihydro-indolo[3,2-d][1]benzazepin-6(5H)-one; (85) 8,10-dichloro-7,12-dihydro-indolo-[3,2-d][1]benzazepin-6(5H)-one; (86) 9-cyano-7,12-dihydro-indolo[3,2-d][1]benzazepin-6(5H)-one; (87) 9-bromo-7,12-dihydro-5-methyl-indolo[3,2-d][1]benzazepin-6(5H)-one; (88) 5-benzyl-9-bromo-7,12-dihydro-5-methyl-indolo[3,2-d][1]-benzazepin-6(5H)-one; (89) 9-bromo-7,12-dihydro-12-methyl-indolo-[3,2-d][1]benzazepin-6(5H)-one; (90) 9-bromo-12-ethyl-7,12-dihydro-indolo[3,2-d][1]benzazepin-6(5H)-one; (91) 9-bromo-7,12-dihydro-12-(2-propenyl)-indolo[3,2-d][1]benzazepin-6(5H)-one; (92) 7,12-dihydro-9-methyl-indolo[3,2-d][1]-benzazepin-6(5H)-one; (93) 7,12-dihydro-9-methoxy-indolo[3,2-d][1]benzazepin-6(5H)-one; (94) 9-fluoro-7,12-dihydro-12-(2-propenyl)-indolo[3,2-d][1]benzazepin-6(5H)-one; (95) 11-bromo-7,12-dihydro-indolo[3,2-d][1]benzazepin-6(5H)-one; (96) 9-bromo-7,12-dihydro-2-(methyliminoamine)-indolo[3,2-d][1]benzazepin-6(5H)-one; (97) 9-bromo-7,12-dihydro-2-(carboxylic acid)indolo[3,2-d][1]benzazepin-6(5H)-one; (98) 9-bromo-7,12-dihydro-10-hydroxy-indolo[3,2-d][1]benzazepin-6(5H)-one; (99) 9-bromo-7,12-dihydro-11-hydroxymethyl-indolo[3,2-d][1]-benzazepin-6(5H)-one; (100) 7,12-dihydro-4-hydroxy-indolo[3,2-d][1]benzazepin-6(5H)-one; (101) 7,12-dihydro-2,3-dihydroxy-indolo[3,2-d][1]benzazepin-6(5H)-one; (102) 2,3-dimethoxy-9-nitro-7,12-dihydro-indolo[3,2-d][1]benzazepin-6(5H)-one; (103) 9-cyano-7,12-dihydro-indolo[3,2-d][1]benzazepin-6(5H)-one; (104) 2,3-dimethoxy-9-cyano-7,12-dihydro-indolo[3,2-d][1]benzazepin-6(5H)-one; (105) 9-nitro-7,12-dihydro-indolo[3,2-d][1]benzazepin-6(5H)-one; (106) 3-(6-oxo-9-trifluoromethyl-5,6,7,12-tetrahydro-indolo[3,2-d][1]benzazepin-2-yl)-propionitrile; (107) 2-bromo-9-nitro-7,12-dihydro-indolo[3,2-d][1]benzazepin-6(5H)-one; (108) 3-(6-oxo-9-trifluoromethyl-5,6,7,12-tetrahydro-indolo[3,2-d][1]benzazepin-2-yl)acrylonitrile; (109) 2-(3-hydroxy-1-propinyl)-9-trifluoromethyl-7,12-dihydro-indolo[3,2-d][1]benzazepin-6(5H)-one; (110) 2-iodo-9-bromo-7,12-dihydro-indolo[3,2-d][1]benzazepin-6(5H)-one; (111) 2-(3-oxo-1-butenyl)-9-trifluoromethyl-7,12-tetrahydro-indolo[3,2-d][1]benzazepin-6(5H)-one; (112) 8-chloro-6,11-dihydro-thieno[3′,2′:2,3]azepino[4,5-b]indol-5(4H)-one; (113) 2-iodo-9-trifluoromethyl-7,12-dihydro-indolo[3,2-d][1]-benzazepin-6(5H)-one; (114) 7,12-dihydro-pyrido[3′,2′:4,5]-pyrrolo[3,2-d][1]benzazepin-6(5H)-one; (115) 11-methyl-7,12-dihydro-indolo[3,2-d][1]-benzazepin-6(5H)-one; (116) 2-[2-(1-hydroxycyclohexyl)-ethinyl]-9-trifluoromethyl-7,12-dihydro-indolo-[3,2-d][1]benzazepin-6(5H)-one; (117) 2-cyano-7,12-dihydro-indolo[3,2-d][1]benzazepin-6(5H)-one; (118) 2-iodo-7,12-dihydro-indolo[3,2-d][1]benzazepin-6(5H)-one; (119) 11-ethyl-7,12-dihydro-indolo[3,2-d][1]benzazepin-6(5H)-one; (120) 8-methyl-6,11-dihydro-thieno[3′,2′:2,3]azepino[4,5-b]indol-5(4H)-one; (121) 3-(6-oxo-9-trifluoromethyl-5,6,7,12-tetrahydro-indolo[3,2-d][1]benzazepin-2-yl)acrylic acid methyl ester; (122) 9-cyano-7,12-dihydro-indolo[3,2-d][1]benzazepin-6(5H)-one; (123) 9-bromo-7,12-dihydro-2,3-dimethoxy-indolo[3,2-d][1]benzazepin-6(5H)-one; (124) 2-bromo-7,12-dihydro-9-trifluoromethyl-indolo[3,2-d][1]benzazepin-6(5H)-one; (125) 7,12-dihydro-2,3-dimethoxy-9-trifluoromethyl-indolo[3,2-d][1]benzazepin-6(5H)-one; (126) 2,9-dibromo-7,12-dihydro-indolo[3,2-d][1]-benzazepin-6(5H)-one; (127) 7,12-dihydro-9-trifluoromethyl-indolo-[3,2-d][1]benzazepin-6(5H)-one; (128) 9-chloro-7,12-dihydro-indolo[3,2-d][1]benzazepin-6(5H)-one; (129) 8-bromo-6,11-dihydro-thieno[3′,2′:2,3]azepino[4,5-b]indole-5(4H)-one; (130) 7,12-dihydro-9-methoxy-indolo[3,2-d][1]benzazepin-6(5H)-one; (131) 10-bromo-7,12-dihydro-indolo[3,2-d][1]benzazepin-6(5H)-one; (132) 11-bromo-7,12-dihydro-indolo[3,2-d][1]-benzazepin-6(5H)-one; (133) 11-chloro-7,12-dihydro-indolo[3,2-d]-[1]benzazepin-6(5H)-one; (134) 9-fluoro-7,12-dihydro-indolo-[3,2-d][1]benzazepin-6(5H)-one; (135) 9-methyl-7,12-dihydro-indolo[3,2-d][1]benzazepin-6(5H)-one; (136) 9-bromo-7,12-dihydro-indolo[3,2-d][1]benzazepin-6(5H)-thione; (137) 8,10-dichloro-7,12-dihydro-indolo[3,2-d][1]benzazepin-6(5H)-one; (138) 9-bromo-7,12-dihydro-12-(2-hydroxyethyl)-indolo[3,2-d][1]-benzazepin-6(5H)-one; (139) 9-bromo-7,12-dihydro-2,3-dihydroxy-indolo[3,2-d][1]benzazepin-6(5H)-one; (140) 2-bromo-7,12-dihydro-indolo[3,2-d][1]benzazepin-6(5H)-one; (141) 7,12-dihydro-2,3-dimethoxy-indolo[3,2-d][1]benzazepin-6(5H)-one; (142) 9-bromo-7,12-dihydro-12-methyl-indolo[3,2-d][1]benzazepin-6(5H)-one; (143) 9-bromo-7,12-dihydro-5-methyloxycarbonylmethyl-indolo[3,2-d][1]benzazepin-6(5H)-one; (144) 7,12-dihydro-indolo[3,2-d][1]benzazepin-6(5H)-one; (145) 9-cyano-7,12-dihydro-indolo[3,2-d][1]benzazepin-6(5H)-one; (146) 9-bromo-7,12-dihydro-2,3-dimethoxy-indolo[3,2-d][1]benzazepin-6(5H)-one; (147) 2-bromo-7,12-dihydro-9-trifluoromethyl-indolo[3,2-d][1]benzazepin-6(5H)-one; (148) 7,12-dihydro-2,3-dimethoxy-9-trifluoromethyl-indolo[3,2-d][1]benzazepin-6(5H)-one; (149) 2,9-dibromo-7,12-dihydro-indolo[3,2-d][1]-benzazepin-6(5H)-one; (150) 7,12-dihydro-9-trifluoromethyl-indolo-[3,2-d][1]benzazepin-6(5H)-one; (151) 9-chloro-7,12-dihydro-indolo[3,2-d][1]benzazepin-6(5H)-one; (152) 8-bromo-6,11-dihydro-thieno[3′,2′:2,3]azepino[4,5-b]indol-5(4H)-one; (153) 7,12-dihydro-9-methoxy-indolo[3,2-d][1]benzazepin-6(5H)-one; (154) 9-bromo-7,12-dihydro-indolo[3,2-d][1]benzazepin-6(5H)-one; (155) 9-chloro-7,12-dihydro-indolo[3,2-d][1]benzazepin-6(5H)-one; (156) 11-chloro-7,12-dihydro-indolo[3,2-d][1]benzazepin-6(5H)-one; (157) 10-bromo-7,12-dihydro-indolo[3,2-d][1]benzazepin-6(5H)-one; (158) 8-bromo-6,11-dihydro-thieno[3′,2′:2,3]azepino-[4,5-b]indol-5(4H)-one; (159) 9-bromo-7,12-dihydro-4-methoxy-indolo[3,2-d][1]benzazepin-6(5H)-one; (160) 9-bromo-7,12-dihydro-4-hydroxy-indolo[3,2-d][1]benzazepin-6(5H)-one; (161) 7,12-dihydro-4-methoxy-indolo[3,2-d][1]benzazepin-6(5H)-one; (162) 9-bromo-7,12-dihydro-2,3-dimethoxy-indolo[3,2-d][1]benzazepin-6(5H)-one; (163) 9-bromo-7,12-dihydro-2,3-di-hydroxy-indolo[3,2-d][1]benzazepin-6(5H)-one; (164) 7,12-dihydro-2,3-dimethoxy-indolo[3,2-d][1]benzazepin-6(5H)-one; (165) 7,12-dihydro-9-trifluormethyl-indolo[3,2-d][1]benzazepin-6(5H)-one; (166) 7,12-dihydro-2,3-dimethoxy-9-trifluoromethyl-indolo[3,2-d][1]benzazepin-6-(5H)-one; (167) 2-bromo-7,12-dihydro-9-trifluoromethyl-indolo[3,2-d][1]benzazepin-6(5H)-one; (168) 9-bromo-7,12-dihydro-indolo[3,2-d][1]benzazepin-6(5H)-thione; (169) 9-bromo-5,12-bis-(t-butyloxycarbonyl)-7,12-dihydro-indolo[3,2-d][1]benzazepin-6(5H)-one; (170) 9-bromo-12-(t-butyloxycarbonyl)-7,12-dihydro-indolo[3,2-d][1]benzazepin-6-(5H)-one: (171) 9-bromo-5,7-bis-(t-butyloxycarbonyl)-7,12-dihydro-indolo[3,2-d][1]benzazepin-6(5H)-one; (172) 9-bromo-5,7,12-tri-(t-butyloxycarbonyl)-7,12-dihydro-indolo[3,2-d][1]benzazepin-6(5H)-one; (173) 9-bromo-7,12-dihydro-5-methyloxycarbonylmethyl-indolo[3,2-d][1]benzazepin-6(5H)-one; (174) 9-bromo-7,12-dihydro-12-methyloxycarbonylmethyl-indolo[3,2-d][1]benzazepin-6(5H)-one; (175) 9-bromo-7,12-dihydro-12-(2-hydroxyethyl)-indolo[3,2-d][1]benzazepin-6-(5H)-one: (176) 2,9-dibromo-7,12-dihydro-indolo[3,2-d][1]benzazepin-6(5H)-one; (177) 8,10-dichloro-7,12-dihydro-indolo-[3,2-d][1]benzazepin-6(5H)-one: (178) 9-cyano-7,12-dihydro-indolo[3,2-d][1]benzazepin-6(5H)-one: (179) 9-bromo-7,12-dihydro-5-methyl-indolo[3,2-d][1]benzazepin-6(5H)-one; (180) 5-benzyl-9-bromo-7,12-dihydro-5-methyl-indolo[3,2-d][1]-benzazepin-6(5H)-one; (181) 9-bromo-7,12-dihydro-12-methyl-indolo-[3,2-d][1]benzazepin-6(5H)-one; (182) 9-bromo-12-ethyl-7,12-dihydro-indolo[3,2-d][1]benzazepin-6(5H)-one; (183) 9-bromo-7,12-dihydro-12-(2-propenyl)-indolo[3,2-d][1]benzazepin-6(5H)-one; (184) 7,12-dihydro-9-methyl-indolo[3,2-d][1]-benzazepin-6(5H)-one; (185) 7,12-dihydro-9-methoxy-indolo[3,2-d][1]benzazepin-6(5H)-one; (186) 9-fluoro-7,12-dihydro-12-(2-propenyl)-indolo[3,2-d][1]benzazepin-6(5H)-one; (187) 11-bromo-7,12-dihydro-indolo[3,2-d][1]benzazepin-6(5H)-one; (188) 9-bromo-7,12-dihydro-2-(methyliminoamine)-indolo[3,2-d][1]benzazepin-6(5H)-one; (189) 9-bromo-7,12-dihydro-2-(carboxylic acid)-indolo[3,2-d][1]benzazepin-6(5H)-one; (190) 9-bromo-7,12-dihydro-10-hydroxy-indolo[3,2-d][1]benzazepin-6(5H)-one: (191) 9-bromo-7,12-dihydro-11-hydroxymethyl-indolo[3,2-d][1]-benzazepin-6(5H)-one: (192) 7,12-dihydro-4-hydroxy-indolo[3,2-d][1]benzazepin-6(5H)-one; (193) 7,12-dihydro-2,3-dihydroxy-indolo[3,2-d][1]benzazepin-6(5H)-one; (194) 2,3-dimethoxy-9-nitro-7,12-dihydro-indolo[3,2-d][1]benzazepin-6(5H)-one; (195) 9-cyano-7,12-dihydro-indolo[3,2-d][1]benzazepin-6(5H)-one; (196) 2,3-dimethoxy-9-cyano-7,12-dihydro-indolo[3,2-d][1]benzazepin-6(5H)-one; (197) 9-nitro-7,12-dihydro-indolo[3,2-d][1]benzazepin-6(5H)-one; (198) 3-(6-oxo-9-trifluoromethyl-5,6,7,12-tetrahydro-indolo[3,2-d][1]benzazepin-2-yl)-propionitrile; (199) 2-bromo-9-nitro-7,12-dihydro-indolo[3,2-d][1]benzazepin-6(5H)-one; (200) 3-(6-oxo-9-trifluoromethyl-5,6,7,12-tetrahydro-indolo[3,2-d][1]benzazepin-2-yl)acrylonitrile; (201) 2-(3-hydroxy-1-propinyl)-9-trifluoromethyl-7,12-dihydro-indolo[3,2-d][1]benzazepin-6(5H)-one; (202) 2-iodo-9-bromo-7,12-dihydro-indolo[3,2-d][1]benzazepin-6(5H)-one; (203) 2-(3-oxo-1-butenyl)-9-trifluoromethyl-7,12-tetrahydro-indolo[3,2-d][1]benzazepin-6(5H)-one; (204) 8-chloro-6,11-dihydro-thieno[3′,2′:2,3]azepino[4,5-b]indol-5(4H)-one; (205) 2-iodo-9-trifluoromethyl-7,12-dihydro-indolo[3,2-d][1]-benzazepin-6(5H)-one; (206) 7,12-dihydro-pyrido[3′,2′:4,5]-pyrrolo[3,2-d][1]benzazepin-6(5H)-one; (207) 11-methyl-7,12-dihydro-indolo[3,2-d][1]-benzazepin-6(5H)-one; (208) 2-[2-(1-hydroxycyclohexyl)-ethinyl]-9-trifluoromethyl-7,12-dihydro-indolo-[3,2-d][1]benzazepin-6(5H)-one; (209) 2-cyano-7,12-dihydro-indolo[3,2-d][1]benzazepin-6(5H)-one; (210) 2-iodo-7,12-dihydro-indolo[3,2-d][1]benzazepin-6(5H)-one; (211) 11-ethyl-7,12-dihydro-indolo[3,2-d][1]benzazepin-6(5H)-one; (212) 8-methyl-6,11-dihydro-thieno[3′,2′:2,3]azepino[4,5-b]indol-5(4H)-one; (213) 3-(6-oxo-9-trifluoromethyl-5,6,7,12-tetrahydro-indolo[3,2-d][1]benzazepin-2-yl)acrylic acid methyl ester; (214) 9-cyano-7,12-dihydro-indolo[3,2-d][1]benzazepin-6(5H)-one; (215) 9-bromo-7,12-dihydro-2,3-dimethoxy-indolo[3,2-d][1]benzazepin-6(5H)-one; (216) 2-bromo-7,12-dihydro-9-trifluoromethyl-indolo[3,2-d][1]benzazepin-6(5H)-one; (217) 7,12-dihydro-2,3-dimethoxy-9-trifluoromethyl-indolo[3,2-d][1]benzazepin-6(5H)-one; (218) 2,9-dibromo-7,12-dihydro-indolo[3,2-d][1]-benzazepin-6(5H)-one; (219) 7,12-dihydro-9-trifluoromethyl-indolo-[3,2-d][1]benzazepin-6(5H)-one; (220) 9-chloro-7,12-dihydro-indolo[3,2-d][1]benzazepin-6(5H)-one; (221) 8-bromo-6,11-dihydro-thieno[3′,2′:2,3]azepino[4,5-b]indole-5(4H)-one: (222) 7,12-dihydro-9-methoxy-indolo[3,2-d][1]benzazepin-6(5H)-one; (223) 10-bromo-7,12-dihydro-indolo[3,2-d][1]benzazepin-6(5H)-one; (224) 11-bromo-7,12-dihydro-indolo[3,2-d][1]-benzazepin-6(5H)-one; (225) 11-chloro-7,12-dihydro-indolo[3,2-d]-[1]benzazepin-6(5H)-one: (226) 9-fluoro-7,12-dihydro-indolo-[3,2-d][1]benzazepin-6(5H)-one; (227) 9-methyl-7,12-dihydro-indolo[3,2-d][1]benzazepin-6(5H)-one; (228) 9-bromo-7,12-dihydro-indolo[3,2-d][1]benzazepin-6(5H)-thione; (229) 8,10-dichloro-7,12-dihydro-indolo[3,2-d][1]benzazepin-6(5H)-one: (230) 9-bromo-7,12-dihydro-12-(2-hydroxyethyl)-indolo[3,2-d][1]-benzazepin-6(5H)-one; (231) 9-bromo-7,12-dihydro-2,3-dihydroxy-indolo[3,2-d][1]benzazepin-6(5H)-one; (232) 2-bromo-7,12-dihydro-indolo[3,2-d][1]benzazepin-6(5H)-one; (233) 7,12-dihydro-2,3-dimethoxy-indolo[3,2-d][1]benzazepin-6(5H)-one; (234) 9-bromo-7,12-dihydro-12-methyl-indolo[3,2-d][1]benzazepin-6(5H)-one; (235) 9-bromo-7,12-dihydro-5-methyloxycarbonylmethyl-indolo[3,2-d][1]benzazepin-6(5H)-one; (236) 7,12-dihydro-indolo[3,2-d][1]benzazepin-6(5H)-one; (237) 9-cyano-7,12-dihydro-indolo[3,2-d][1]benzazepin-6(5H)-one; (238) 9-bromo-7,12-dihydro-2,3-dimethoxy-indolo[3,2-d][1]benzazepin-6(5H)-one; (239) 2-bromo-7,12-dihydro-9-trifluoromethyl-indolo[3,2-d][1]benzazepin-6(5H)-one; (240) 7,12-dihydro-2,3-dimethoxy-9-trifluoromethyl-indolo[3,2-d][1]benzazepin-6(5H)-one; (241) 2,9-dibromo-7,12-dihydro-indolo[3,2-d][1]-benzazepin-6(5H)-one; (242) 7,12-dihydro-9-trifluormethyl-indolo-[3,2-d][1]benzazepin-6(5H)-one; (243) 9-chloro-7,12-dihydro-indolo[3,2-d][1]benzazepin-6(5H)-one: (244) 8-bromo-6,11-dihydro-thieno[3′,2′:2,3]azepino[4,5-b]indol-5(4H)-one; (245) 7,12-dihydro-9-methoxy-indolo[3,2-d][1]benzazepin-6(5H)-one; (246) 9-bromo-7,12-dihydro-indolo[3,2-d][1]benzazepin-6(5H)-one (247) 6-bromoindirubin; (248) 6,6′-dibromoindirubin-3-oxime; (249) 6-bromoindirubin-3′-methoxime; (250) 6-bromo-5-methyl-indirubin; (251) 6-bromo-5-aminoindirubin; (252) 6-bromo-5-methyl-indirubin-3′-oxime; (253) 6-bromo-indirubin-3′-acetoxime; (254) 5-amino-indirubin; (255) 5-amino-indirubin-3′-oxime; (256) 1-methylindirubin; (257) N-1-methylisoindigo; (258) indirubin-5-sulfone-(2-hydroxyethyl)amide; (259) 5-ethylindirubin; (260) 5-isopropylindirubin; (261) 5-n-propylindirubin; (262) 5-carboxymethylindirubin; (263) 5-[2-(piperazin-1-yl)-ethan-2-on-1-yl]indirubin; (264) 5-[2-(morpholin-1-yl)-ethan-2-on-1-yl]indirubin; (265) N-(2-aminoethyl)-2-[3-(3′-oxo-(2′H,3′H)indol-2′-ylidene)-(2H,3H)indol-2-one-5-yl]acetamide; (266) N-methyl-2-[3-(3′-oxo-(2′H,3′H)indol-2′-ylidene)-(2H,3H)indol-2-one-5-yl]acetamide; (267) N,N-dimethyl-2-[3-(3′-oxo-(2′H,3′H)indol-2′-ylidene)-(2H,3H)indol-2-one-5-yl]acetamide; (268) 2-{2-[3′-oxo-(2′H,3′H)indol-2′-ylidene)-(2H,3H)indol-2-one-5-yl]-acetylamino}-acetic acid; (269) methyl-2-{2-[3′-oxo-(2′H,3′H)indol-2′-ylidene)-(2H,3H)indol-2-one-5-yl]-acetylamino}-acetate; (270) [3′-oxo-(2′H,3′H)indol-2′-ylidene)-(2H,3H)indol-2-one-5-yl]-methylphosphonic acid; (271) diethyl-[3′-oxo-(2′H,3′H)indol-2′-ylidene)-(2H,3H)indol-2-one-5-yl]-methylphosphonate; (272) 5-acetylaminoindirubin; (274) [3′-oxo-(2′H,3′H)indol-2′-ylidene)-(2H,3H)indol-2-one-5-yl]-succinamic acid; (275) 2-amino-N-[3′-oxo-(2′H,3′H)indol-2′-ylidene)-(2H,3H)indol-2-one-5-yl]-acetamide; (276) 2-amino-N-[3′-oxo-(2′H,3′H)indol-2′-ylidene)-(2H,3H)indol-2-one-5-yl]-propionamide; (277) 5-(2-aminoethyl)-aminoindirubin; (278) 5-(2-hydroxyethyl)-aminoindirubin; (279) indirubin-5-sulfonic acid-(piperazin-1-yl-amide); (280) indirubin-5-sulfonic acid-(morpholin-1-yl-amide); (281) methyl-2-{[3′-oxo-(2′H,3′H)indol-2′-ylidene)-(2H,3H)indol-2-one-5-yl]-sulfonamidyl}-acetate; (282) 5-methylindirubin-3′-monooxime; (283) 5-ethylindirubin-3′-monooxime; (284) 5-isopropylindirubin-3′-monooxime; (285) 5-aminoindirubin-3′-monooxime; (286) 5-acetylaminoindirubin-3′-monooxime; (287) 2-amino-N-[3-(3′-hydroxyimino)(2′H,3′H)indol-2′-ylidene)-(2H,3H)indol-2-one-5-yl]-acetamide; (288) 3-[3′-(iminooxy-O-(2-hydroxyethyl)-(2′H,3′H)indol-2′-ylidene]-(2H,3H)indol-2-one; (289) 3-[3′-(iminooxy-O-(3-hydroxypropyl)-(2′H,3′H)indol-2′-ylidene]-(2H,3H)indol-2-one; (290) 3-[3′-(iminooxy-O-(2-(2-hydroxyethoxy)ethyl)-(2′H,3′H)indol-2′-ylidene]-(2H,3H)indol-2-one; (291) 3-[3′-(iminooxy-O-(2-(2-hydroxy-2-methyl)propyl)-(2′H,3′H)indol-2′-ylidene]-(2H,3H)indol-2-one; (292) 2-{O-[2′-(2-oxo-(2H,3H)indol-3-ylidene)-2′H,3′H-indol-3′-ylidene]aminoxy}acetic acid sodium salt; (293) 3-{O-[2′-(2-oxo-(2H,3H)indol-3-ylidene)-2′H,3′H-indol-3′-ylidene]aminoxy}propionic acid sodium salt; (294) 4-{O-[2′-(2-oxo-(2H,3H)indol-3-ylidene)-2′H,3′H-indol-3′-ylidene]aminoxy}butyric acid sodium salt; (295) 5-{O-[2′-(2-oxo-(2H,3H)indol-3-ylidene)-2′H,3′H-indol-3′-ylidene]aminoxy}pentanoic acid sodium salt; (296) 3-[3′-iminooxy-O-carbethoxy)-(2′H,3′H)-indol-2′-ylidene]-(2H,3H)indol-2-one; (297) ethyl-2-{O-2′-(2-oxo-(2H,3H)indol-3-ylidene-(2′H,3′H)-indol-3′-ylidene]-aminooxy}-acetate; (298) 3-[3′-iminooxy-O—(N,N)-dimethylcarbamoyl)]-(2′H,3′H)-indol-2′-ylidene]-(2H,3H)indol-2-one); (299) 3′-oximido-7-azaindirubin; (300) 7-azaindirubin-3′-oxime ether; (301) 1-methyl-5-azaindirubin; (302) 1-benzyl-5′-chloro-5-azaindirubin; (303) 1-butyl-5-azaindirubin-3′-oxime; (304) 1-butyl-5-azaindirubin-3′-oxime O-methyl ether; (305) 1-isopropyl-5-azaisoindigo; (306) 1-methyl-7-azaindirubin; (307) 1-benzyl-5′-bromo-7-azaindirubin; (308) 1-butyl-7-azaindirubin-3′-oxime; (309) 1-butyl-7-azaindirubin-3′-oxime O-methyl ether; (310) 1-isopropyl-7-azaisoindigo; (311) 2-methyl-7-[1,2-dihydro-2-oxo-3H-indol-(Z)-3-ylidene]-4,5,6,7-tetrahydro-1H-indole-3-carboxylic acid; (312) 2-methyl-7-[1,2-dihydro-5-fluoro-2-oxo-3H-indol-(Z)-3-ylidene]-4,5,6,7-tetrahydro-1H-indole-3-carboxylic acid; (313) 2-methyl-7-[1,2-dihydro-5-chloro-2-oxo-3H-indol-(Z)-3-ylidene]-4,5,6,7-tetrahydro-1H-indole-3-carboxylic acid; (314) 2-methyl-7-[1,2-dihydro-5-methyl-2-oxo-3H-indol-(Z)-3-ylidene]-4,5,6,7-tetrahydro-1H-indole-3-carboxylic acid; (315) ethyl 2-methyl-7-[1,2-dihydro-5-fluoro-2-oxo-3H-indol-(Z)-3-ylidene]-4,5,6,7-tetrahydro-1H-indole-3-carboxylate; (316) N-(2-(diethylamino)ethyl)-2-methyl-7-[1,2-dihydro-5-fluoro-2-oxo-3H-indol-(Z)-3-ylidene]-4,5,6,7-tetrahydro-1H-indole-3-carboxamide; (317) N-(2-(diethylamino)ethyl)-2-methyl-7-[1,2-dihydro-5-chloro-2-oxo-3H-indol-(Z)-3-ylidene]-4,5,6,7-tetrahydro-1H-indole-3-carboxamide; (318) N-(2-(diethylamino)ethyl)-2-methyl-7-[1,2-dihydro-5-methyl-2-oxo-3H-indol-(Z)-3-ylidene]-4,5,6,7-tetrahydro-1H-indole-3-carboxamide; (319) N-(2-(diethylamino)ethyl)-2-methyl-7-[1,2-dihydro-2-oxo-3H-indol-(Z)-3-ylidene]-4,5,6,7-tetrahydro-1H-indole-3-carboxamide; (320) N-(2-(dimethylamino)ethyl)-2-methyl-7-[1,2-dihydro-5-fluoro-2-oxo-3H-indo-I—(Z)-3-ylidene]-4,5,6,7-tetrahydro-1H-indole-3-carboxamide; (321) N-(3-(dimethylamino)propyl)-2-methyl-7-[1,2-dihydro-5-chloro-2-oxo-3H-indol-(Z)-3-ylidene]-4,5,6,7-tetrahydro-1H-indole-3-carboxamide; (322) N-(2-hydroxyethyl)-2-methyl-7-[1,2-dihydro-2-oxo-3H-indol-(Z)-3-ylidene]-4,5,6,7-tetrahydro-1H-indole-3-carboxamide; (323) N-(2-hydroxyethyl)-2-methyl-7-[1,2-dihydro-5-fluoro-2-oxo-3H-indol-(Z)-3-ylidene]-4,5,6,7-tetrahydro-1H-indole-3-carboxamide (324); (325) 2-methyl-3-(morpholine-4-carbonyl)-7-[1,2-dihydro-5-fluoro-2-oxo-3H-indol-(Z)-3-ylidene]-4,5,6,7-tetrahydro-1H-indole; (326) 2-methyl-3-(morpholine-4-carbonyl)-7-[1,2-dihydro-5-chloro-2-oxo-3H-indol-(Z)-3-ylidene]-4,5,6,7-tetrahydro-1H-indole; (327) 2-methyl-3-(morpholine-4-carbonyl)-7-[1,2-dihydro-2-oxo-3H-indol-(Z)-3-ylidene]-4,5,6,7-tetrahydro-1H-indole; (328) 2-methyl-3-(4-methylpiperazine-1-carbonyl)-7-[1,2-dihydro-2-oxo-3H-indol-(Z)-3-ylidene]-4,5,6,7-tetrahydro-1H-indole; (329) 2-methyl-3-(4-methylpiperazine-1-carbonyl)-7-[1,2-dihydro-5-fluoro-2-oxo-3H-indol-(Z)-3-ylidene]-4,5,6,7-tetrahydro-1H-indole; (330) N,N,2-trimethyl-7-[1,2-dihydro-2-oxo-3H-indol-(Z)-3-ylidene]-4,5,6,7-tetrahydro-1H-indole-3-carboxamide; (331) N-(2-morpholinoethyl)-2-methyl-7-[1,2-dihydro-5-methyl-2-oxo-3H-indol-(Z)-3-ylidene]-4,5,6,7-tetrahydro-1H-indole-3-carboxamide; (332) N-(2-morpholinoethyl)-2-methyl-7-[1,2-dihydro-5-methyl-2-oxo-3H-indol-(Z)-3-ylidene]-4,5,6,7-tetrahydro-1H-indole-3-carboxamide; (333) N-(2-morpholinoethyl)-2-methyl-7-[1,2-dihydro-2-oxo-3H-indol-(Z)-3-ylidene]-4,5,6,7-tetrahydro-1H-indole-3-carboxamide; (334) N-(2-morpholinoethyl)-2-methyl-7-[1,2-dihydro-5-chloro-2-oxo-3H-indol-(Z)-3-ylidene]-4,5,6,7-tetrahydro-1H-indole-3-carboxamide; (345) N-(2-morpholinoethyl)-2-methyl-7-[1,2-dihydro-5-fluoro-2-oxo-3H-indol-(Z)-3-ylidene]-4,5,6,7-tetrahydro-1H-indole-3-carboxamide; (346) N-(3-morpholinopropyl)-2-methyl-7-[1,2-dihydro-5-fluoro-2-oxo-3H-indol-(Z)-3-ylidene]-4,5,6,7-tetrahydro-1H-indole-3-carboxamide; (347) N-(3-morpholinopropyl)-2-methyl-7-[1,2-dihydro-5-bromo-2-oxo-3H-indol-(Z)-3-ylidene]-4,5,6,7-tetrahydro-1H-indole-3-carboxamide; (348) N-(2-morpholinoethyl)-2-methyl-7-[1,2-dihydro-7-fluoro-2-oxo-3H-indol-(Z)-3-ylidene]-4,5,6,7-tetrahydro-1H-indole-3-carboxamide; (349) N-(2-(pyrrolidin-1-yl)ethyl)-2-methyl-7-[1,2-dihydro-2-oxo-3H-indol-(Z)-3-ylidene]-4,5,6,7-tetrahydro-1H-indole-3-carboxamide; (350) N-(2-(piperidin-1-yl)ethyl)-2-methyl-7-[1,2-dihydro-2-oxo-3H-indol-(Z)-3-ylidene]-4,5,6,7-tetrahydro-1H-indole-3-carboxamide; (351) N-(2-(piperidin-1-yl)ethyl)-2-methyl-7-[1,2-dihydro-5-fluoro-2-oxo-3H-indol-(Z)-3-ylidene]-4,5,6,7-tetrahydro-1H-indole-3-carboxamide; (352) N-(2-(pyrrolidin-1-yl)ethyl)-2-methyl-7-[1,2-dihydro-5-fluoro-2-oxo-3H-indol-(Z)-3-ylidene]-4,5,6,7-tetrahydro-1H-indole-3-carboxamide; (353) N-(3-(pyrrolidin-1-yl)propyl)-2-methyl-7-[1,2-dihydro-5-fluoro-2-oxo-3H-indol-(Z)-3-ylidene]-4,5,6,7-tetrahydro-1H-indole-3-carboxamide; (354) N-(3-(4-methylpiperazin-1-yl)propyl)-2-methyl-7-[1,2-dihydro-5-fluoro-2-oxo-3H-indol-(Z)-3-ylidene]-4,5,6,7-tetrahydro-1H-indole-3-carboxamide; (355) N-(3-(pyrrolidin-1-yl)propyl)-2-methyl-7-[1,2-dihydro-5-bromo-2-oxo-3H-indol-(Z)-3-ylidene]-4,5,6,7-tetrahydro-1H-indole-3-carboxamide; (356) N-(2-(piperidin-1-yl)ethyl)-2-methyl-7-[1,2-dihydro-6-chloro-2-oxo-3H-indol-(Z)-3-ylidene]-4,5,6,7-tetrahydro-1H-indole-3-carboxamide; (357) N-(3-(pyrrolidin-1-yl)propyl)-2-methyl-7-[1,2-dihydro-4-fluoro-2-oxo-3H-indol-(Z)-3-ylidene]-4,5,6,7-tetrahydro-1H-indole-3-carboxamide; (358) N-(3-(pyrrolidin-1-yl)propyl)-2-methyl-7-[1,2-dihydro-7-fluoro-2-oxo-3H-indol-(Z)-3-ylidene]-4,5,6,7-tetrahydro-1H-indole-3-carboxamide; (359) N-(2-(diethylamino)ethyl)-2-methyl-7-[1,2-dihydro-5,7-dimethyl-2-oxo-3H-indol-(Z)-3-ylidene]-4,5,6,7-tetrahydro-1H-indole-3-carboxamide; (360) N-(2-(diethylamino)ethyl)-2-methyl-7-[N-isopropyl-1,2-dihydro-2-oxo-3H-indol-5-sulfonamide-(Z)-3-ylidene]-4,5,6,7-tetrahydro-1H-indole-3-carboxamide; (361) N-(2-(diethylamino)ethyl)-2-methyl-7-[1,2-dihydro-5-bromo-2-oxo-3H-indol-(Z)-3-ylidene]-4,5,6,7-tetrahydro-1H-indole-3-carboxamide; (362) N-(2-(diethylamino)ethyl)-2-methyl-7-[1,2-dihydro-5-nitro-2-oxo-3H-indol-(Z)-3-ylidene]-4,5,6,7-tetrahydro-1H-indole-3-carboxamide; (363) N-(3-(dimethylamino)propyl)-2-methyl-7-[1,2-dihydro-5-fluoro-2-oxo-3H-indol-(Z)-3-ylidene]-4,5,6,7-tetrahydro-1H-indole-3-carboxamide; (364) N-(2-(diethylamino)ethyl)-2-methyl-7-[1,2-dihydro-5-methoxy carbonyl-2-oxo-3H-indol-(Z)-3-ylidene]-4,5,6,7-tetrahydro-1H-indole-3-carboxamide; (365) N-(2-(diethylamino)ethyl)-2-methyl-7-[1,2-dihydro-7-fluoro-2-oxo-3H-indol-(Z)-3-ylidene]-4,5,6,7-tetrahydro-1H-indole-3-carboxamide; (366) N-(2-(diethylamino)ethyl)-2-methyl-7-[N-(4-fluorophenyl)-1,2-dihydro-2-oxo-3H-indol-5-sulfonamide-(Z)-3-ylidene]-4,5,6,7-tetrahydro-1H-indole-3-carboxamide); (367) N-(2-(diethylamino)ethyl)-2-methyl-7-[5-(piperidin-1-ylsulfonyl)-1,2-dihydro-2-oxo-3H-indol-5-sulfonamide-(Z)-3-ylidene]-4,5,6,7-tetrahydro-1H-indole-3-carboxamide; (368) N-(3-(diethylamino)propyl)-2-methyl-7-[1,2-dihydro-5-fluoro-2-oxo-3H-indo-1-(Z)-3-ylidene]-4,5,6,7-tetrahydro-1H-indole-3-carboxamide; (369) N-(2-(diethylamino)ethyl)-2-methyl-7-[1,2-dihydro-5-carboxyl-2-oxo-3H-indol-(Z)-3-ylidene]-4,5,6,7-tetrahydro-1H-indole-3-carboxamide; (370) N-(2-(diethylamino)ethyl)-2-methyl-7-[1,2-dihydro-5-carboxyl-2-oxo-3H-indol-(Z)-3-ylidene]-4,5,6,7-tetrahydro-1H-indole-3-carboxamide; (371) N-(2-(diethylamino)ethyl)-2-methyl-7-[1,2-dihydro-6-chloro-2-oxo-3H-indol-(Z)-3-ylidene]-4,5,6,7-tetrahydro-1H-indole-3-carboxamide; 372) N-(3-(diethylamino)propyl)-2-methyl-7-[1,2-dihydro-6-chloro-2-oxo-3H-indol-(Z)-3-ylidene]-4,5,6,7-tetrahydro-1H-indole-3-carboxamide; (373) N-(3-(diethylamino)propyl)-2-methyl-7-[1,2-dihydro-5-bromo-2-oxo-3H-indol-(Z)-3-ylidene]-4,5,6,7-tetrahydro-1H-indole-3-carboxamide; (374) N-(2-(diethylamino)ethyl)-2-methyl-7-[1,2-dihydro-4-fluoro-2-oxo-3H-indol-(Z)-3-ylidene]-4,5,6,7-tetrahydro-1H-indole-3-carboxamide; (375) N-(2-(diethylamino)ethyl)-2-methyl-7-[5-(pyrrolidine-1-carbonyl)-1,2-dihydro-2-oxo-3H-indol-(Z)-3-ylidene]-4,5,6,7-tetrahydro-1H-indole-3-carboxamide; (376) N-(2-(diethylamino)ethyl)-2-methyl-7-[N-(4-fluorophenyl)-5-carboxamide-1,2-dihydro-2-oxo-3H-indol-(Z)-3-ylidene]-4,5,6,7-tetrahydro-1H-indole-3-carboxamide; (377) N-(3-(diethylamino)propyl)-2-methyl-7-[1,2-dihydro-7-fluoro-2-oxo-3H-indol-(Z)-3-ylidene]-4,5,6,7-tetrahydro-1H-indole-3-carboxamide; (378) N-(2-(diethylamino)ethyl)-2-methyl-7-[1,2-dihydro-5-methoxy-2-oxo-3H-indol-(Z)-3-ylidene]-4,5,6,7-tetrahydro-1H-indole-3-carboxamide; (379) N-(2-(diethylamino)ethyl)-2-methyl-7-[1,2-dihydro-5-trifluoromethoxy-2-oxo-3H-indol-(Z)-3-ylidene]-4,5,6,7-tetrahydro-1H-indole-3-carboxamide; (380) N-(2-(diethylamino)ethyl)-2-methyl-7-[N-methyl-1,2-dihydro-2-oxo-3H-indol-5-sulfonamide-(Z)-3-ylidene]-4,5,6,7-tetrahydro-1H-indole-3-carboxamide; (381) N-(2-(pyridin-2-yl)ethyl)-2-methyl-7-[1,2-dihydro-5-fluoro-2-oxo-3H-indol-(Z)-3-ylidene]-4,5,6,7-tetrahydro-1H-indole-3-carboxamide; (382) N-(2-(dimethylamino)ethyl)-N,2-dimethyl-7-[1,2-dihydro-5-fluoro-2-oxo-3H-indol-(Z)-3-ylidene]-4,5,6,7-tetrahydro-1H-indole-3-carboxamide; (383) N-(2-(dimethylamino)ethyl)-N,2-dimethyl-7-[1,2-dihydro-6-chloro-2-oxo-3H-indol-(Z)-3-ylidene]-4,5,6,7-tetrahydro-1H-indole-3-carboxamide; (384) N-benzyl-N,2-dimethyl-7-[1,2-dihydro-5-fluoro-2-oxo-3H-indol-(Z)-3-ylidene]-4,5,6,7-tetrahydro-1H-indole-3-carboxamide; (385) 2-methyl-3-[(S)-2-(pyrrolidin-1-ylmethyl)pyrrolidin-1-carbonyl)]-7-[1,2-dihydro-5-fluoro-2-oxo-3H-indol-(Z)-3-ylidene]-4,5,6,7-tetrahydro-1H-indole; (386) 2-methyl-3-[4-(2-hydroxyethyl)-piperazin-1-carbonyl]-7-[1,2-dihydro-5-fluoro-2-oxo-3H-indol-(Z)-3-ylidene]-4,5,6,7-tetrahydro-1H-indole; (387) 2-methyl-3-(1,4′-bipiperidin-1′-carbonyl)-7-[1,2-dihydro-5-fluoro-2-oxo-3-H-indol-(Z)-3-ylidene]-4,5,6,7-tetrahydro-1H-indole; (388) N-(3-(diethylamino)-2-hydroxypropyl)-2-methyl-7-[1,2-dihydro-2-oxo-3H-indol-(Z)-3-ylidene]-4,5,6,7-tetrahydro-1H-indole-3-carboxamide; (389) N-(3-(diethylamino)-2-hydroxypropyl)-2-methyl-7-[1,2-dihydro-2-oxo-3H-indol-(Z)-3-ylidene]-4,5,6,7-tetrahydro-1H-indole-3-carboxamide; (390) N-(3-(diethylamino)-2-hydroxypropyl)-2-methyl-7-[1,2-dihydro-5-chloro-2-oxo-3H-indol-(Z)-3-ylidene]-4,5,6,7-tetrahydro-1H-indole-3-carboxamide; (391) N-(3-(dimethylamino)-2-hydroxypropyl)-2-methyl-7-[1,2-dihydro-5-fluoro-2-oxo-3H-indol-(Z)-3-ylidene]-4,5,6,7-tetrahydro-1H-indole-3-carboxamide; (392) N-(2-hydroxy-3-morpholinopropyl)-2-methyl-7-[1,2-dihydro-2-oxo-3H-indol-(Z)-3-ylidene]-4,5,6,7-tetrahydro-1H-indole-3-carboxamide; (393) N-(2-hydroxy-3-morpholinopropyl)-2-methyl-7-[1,2-dihydro-5-fluoro-2-oxo-3-H-indol-(Z)-3-ylidene]-4,5,6,7-tetrahydro-1H-indole-3-carboxamide; (394) N-(2-hydroxy-3-morpholinopropyl)-2-methyl-7-[1,2-dihydro-5-chloro-2-oxo-3-H-indol-(Z)-3-ylidene]-4,5,6,7-tetrahydro-1H-indole-3-carboxamide; (395) N-(2-hydroxy-3-(pyrrolidin-1-yl)propyl)-2-methyl-7-[1,2-dihydro-2-oxo-3H-indol-(Z)-3-ylidene]-4,5,6,7-tetrahydro-1H-indole-3-carboxamide; (396) N-(2-hydroxy-3-(pyrrolidin-1-yl)propyl)-2-methyl-7-[1,2-dihydro-5-fluoro-2-oxo-3H-indol-(Z)-3-ylidene]-4,5,6,7-tetrahydro-1H-indole-3-carboxamide; (397) N-(2-hydroxy-3-(pyrrolidin-1-yl)propyl)-2-methyl-7-[1,2-dihydro-5-chloro-2-oxo-3H-indol-(Z)-3-ylidene]-4,5,6,7-tetrahydro-1H-indole-3-carboxamide; (398) N-(2-hydroxy-3-(piperidin-1-yl)propyl)-2-methyl-7-[1,2-dihydro-2-oxo-3H-indol-(Z)-3-ylidene]-4,5,6,7-tetrahydro-1H-indole-3-carboxamide; (399) N-(2-hydroxy-3-(piperidin-1-yl)propyl)-2-methyl-7-[1,2-dihydro-5-fluoro-2-oxo-3H-indol-(Z)-3-ylidene]-4,5,6,7-tetrahydro-1H-indole-3-carboxamide; (400) N-(2-hydroxy-3-(piperidin-1-yl)propyl)-2-methyl-7-[1,2-dihydro-5-chloro-2-oxo-3H-indol-(Z)-3-ylidene]-4,5,6,7-tetrahydro-1H-indole-3-carboxamide; (401) N-[2-hydroxy-3-(4-methylpiperazin-1-yl)propyl]-2-methyl-7-[1,2-dihydro-2-oxo-3H-indol-(Z)-3-ylidene]-4,5,6,7-tetrahydro-1H-indole-3-carboxamide; (402) N-[2-hydroxy-3-(4-methylpiperazin-1-yl)propyl]-2-methyl-7-[1,2-dihydro-5-fluoro-2-oxo-3H-indol-(Z)-3-ylidene]-4,5,6,7-tetrahydro-1H-indole-3-carboxamide; (403) N-[2-hydroxy-3-(4-methylpiperazin-1-yl)propyl]-2-methyl-7-[1,2-dihydro-5 chloro-2-oxo-3H-indol-(Z)-3-ylidene]-4,5,6,7-tetrahydro-1H-indole-3-carboxamide; (404) N-[3-(cyclohexyl(methyl)amino)-2-hydroxypropyl]-2-methyl-7-[1,2-dihydro-5-methyl-2-oxo-3H-indol-(Z)-3-ylidene]-4,5,6,7-tetrahydro-1H-indole-3-carboxamide; (405) N-(3-(diethylamino)-2-hydroxypropyl)-2-methyl-7-[1,2-dihydro-5-bromo-2-oxo-3H-indol-(Z)-3-ylidene]-4,5,6,7-tetrahydro-1H-indole-3-carboxamide; (406) N-(2-hydroxy-3-morpholinopropyl)-2-methyl-7-[1,2-dihydro-6-chloro-2-oxo-3-H-indol-(Z)-3-ylidene]-4,5,6,7-tetrahydro-1H-indole-3-carboxamide; (407) N-[3-(cyclohexyl(methyl)amino)-2-hydroxypropyl]-2-methyl-7-[1,2-dihydro-5-fluoro-2-oxo-3H-indol-(Z)-3-ylidene]-4,5,6,7-tetrahydro-1H-indole-3-carboxamide; (408) 5-bromoindirubin-3′-oxime; (409) 7-bromoindirubin-3′-oxime; (410) 7-chloroindirubin-3′-oxime; (411) 7-iodoindirubin-3′-oxime; (412) 7-fluoroindirubin-3′-oxime; (413) 1-methyl-7-bromoindirubin-3′-oxime; (414) (2′Z)-7-fluoroindirubin; (415) (2′Z)-7-chloroindirubin; (416) (2′Z)-7-bromoindirubin; (417) (2′Z)-7-iodoindirubin; (418) (2′Z)-7-fluoro-1-methylindirubin; (419) (2′Z)-7-chloro-1-methylindirubin; (420) (2′Z)-7-bromo-1-methylindirubin; (421) (2′Z)-7-iodo-1-methylindirubin; (422 (2′Z,3′E)-7-fluoroindirubin-3′-oxime; (423) (2′Z,3′E)-7-chloroindirubin-3′-oxime; (424) (2′Z,3′E)-7-bromoindirubin-3′-oxime; (425) (2′Z,3′E)-7-iodoindirubin-3′-oxime; (426) (2′Z,3′E)-7-fluoro-1-methylindirubin-3′-oxime; (427) (2′Z,3′E)-7-chloro-1-methylindirubin-3′-oxime; (428) (2′Z,3′E)-7-bromo-1-methylindirubin-3′-oxime; (429) (2′Z,3′E)-7-iodo-1-methylindirubin-3′-oxime; (430) (2′Z,3′E)-7-fluoroindirubin-3′-acetoxime; (431) (2′Z,3′E)-7-chloroindirubin-3′-acetoxime; (432) (2′Z,3′E)-7-bromoindirubin-3′-acetoxime; (433) (2′Z,3′E)-7-iodoindirubin-3′-acetoxime; (434) (2′Z,3′E)-7-fluoro-1-methylindirubin-3′-acetoxime; (435) (2′Z,3′E)-7-chloro-1-methylindirubin-3′-acetoxime; (436) (2′Z,3′E)-7-bromo-1-methylindirubin-3′-acetoxime; (437) (2′Z,3′E)-7-iodo-1-methylindirubin-3′-acetoxime; (438) (2′Z,3′E)-7-fluoroindirubin-3′-methoxime; (439) (2′Z,3′E)-7-chloroindirubin-3′-methoxime; (440) (2′Z,3′E)-7-bromoindirubin-3′-methoxime: (441) (2′Z,3′E)-7-iodoindirubin-3′-methoxime; (442) (2′Z,3′E)-7-fluoro-1-methylindirubin-3′-methoxime; (443) (2′Z,3′E)-7-chloro-1-methylindirubin-3′-methoxime; (444) (2′Z,3′E)-7-bromo-1-methylindirubin-3′-methoxime, (2′Z,3′E)-7-iodo-1-methylindirubin-3′-methoxime; (445) (2′Z,3′E)-7-bromoindirubin-3′-[O-(2-bromoethyl)-oxime]; (446) (2′Z,3′E)-1-methyl-7-bromoindirubin-3′-[O-(2-bromoethyl)-oxime]; (447) (2′Z,3′E)-7-bromoindirubin-3′-[O-(N,N-diethylcarbamyl)-oxime]; (448) 2′Z,3′E)-1-methyl-7-bromoindirubin-3′-[O-(N,N-diethylcarbamyl)-oxime]; (449) (2′Z,3′E)-7-bromoindirubin-3′-[O-(2-pyrrolidin-1-yl-ethyl)-oxime]; (450) (2′Z,3′E)-1-methyl-7-bromoindirubin-3′-[O-(2-pyrrolidin-1-yl-ethyl)-oxime], (451) (2′Z,3′E)-7-bromoindirubin-3′-[O-(2-morpholin-1-yl-ethyl)-oxime], (452) (2′Z,3′E)-1-methyl-7-bromoindirubin-3′-[O-(2-morpholin-1-yl-ethyl)-oxime]; (453) (2′Z,3′E)-7-bromoindirubin-3′-[O-(2-imidazol-1-yl-ethyl)-oxime]; (454) (2′Z,3′E)-1-methyl-7-bromoindirubin-3′-[O-(2-imidazol-1-yl-ethyl)-oxime]; (455) (2′Z,3′E)-7-bromoindirubin-3′-[O-(2-piperazin-1-yl-ethyl)-oxime]; (456) (2′Z,3′E)-7-bromoindirubin-3′-[O-(2-dimethylaminoethyl)-oxime]; (457) (2′Z,3′E)-1-methyl-7-bromoindirubin-3′-[O-(2-dimethylaminoethyl)-oxime]; (458) (2′Z,3′E)-7-bromoindirubin-3′-[O-(2-diethylaminoethyl)-oxime] (459) (2′Z,3′E)-1-methyl-7-bromoindirubin-3′-[O-(2-diethylaminoethyl)-oxime]; (460) N-(2-hydroxy-3-morpholinopropyl)-2-methyl-7-[1,2-dihydro-5-bromo-2-oxo-3H-indol-(Z)-3-ylidene]-4,5,6,7-tetrahydro-1H-indole-3-carboxamide; (461) 6-bromoindirubin-3′-oxime; (462) (2′Z,3′E)-6-bromoindirubin-3′-[O-(2-bromoethyl)-oxime]; (463) (2′Z,3′E)-6-bromoindirubin-3′-[O-(2-hydroxyethyl)-oxime]; (464) (2′Z,3′E)-6-bromoindirubin-3′-[O-(2,3-dihydroxypropyl)-oxime]; (465) (2′Z,3′E)-6-bromoindirubin-3-[O—(N,N-diethylcarbamyl)-oxime]; (466) (2′Z,3′E)-6-bromoindirubin-3′-[O-(2-dimethylaminoethyl)-oxime]; (467) (2′Z,3′E)-6-bromoindirubin-3-[O-(2-diethylaminoethyl)-oxime]; (468) (2′Z,3′E)-6-bromoindirubin-3′-[O-(2-pyrrolidin-1-ylethyl)-oxime]; (469) (2′Z,3′E)-6-bromoindirubin-3′-[O-(2-morpholin-1-ylethyl)-oxime]; (470) (2′Z,3′E)-6-bromoindirubin-3′-[O-(2-N,N-(2-hydroxyethyl)aminoethyl)-oxime]; (471) (2′Z,3′E)-6-bromoindirubin-3′-(O-{2-N,N-dimethyl, N-(2,3-dihydroxypropyl)amino]ethyl}oxime; (472) (2′Z,3′E)-6-bromoindirubin-3′-[O-(2-piperazin-1-ylethyl)-oxime]; (473) (2′Z,3′E)-6-bromoindirubin-3′-{O-[2-(4-methyl-piperazin-1-yl)ethyl]oxime; (474) (2′Z,3′E)-6-bromoindirubin-3′-O-{2-[4-(2-hydroxyethyl)piperazin-1-yl]ethyl}oxime; (475) (2′Z,3′E)-6-bromoindirubin-3′-O-{2-[4-(2-methoxyethyl)piperazin-1-yl]ethyl}oxime; (476) (2′Z,3′E)-6-bromoindirubin-3′-O—[O-2-{4-[2-(2-hydroxyethoxy)-ethyl]piperazin-1-yl}ethyl)oxime; (477) isoindigo; (478) 5-nitroindirubin-3′-oxime; (479) 5′-bromo-5-nitroindirubin-3′-oxime; (480) 5′-hydroxy-5-nitroindirubin-3′-oxime; (481) 5′-hydroxy-5-chloroindirubin-3′-oxime; (482) 5′-hydroxy-5-fluoroindirubin-3′-oxime; (483) 5′-chloro-5-nitroindirubin-3′-oxime; (484) 5′-methyl-5-nitroindirubin-3′-oxime; (485) indirubin-5-sulfonic acid (2-hydroxyethyl)-amide; (486) (3-[3-(3,4-dihydroxybutoxyamino)-1H-indol-2-yl]indol-2-one); and the salts, solvates, analogues, congeners, bioisosteres, hydrolysis products, metabolites, precursors, and prodrugs thereof (hereinafter “Alternatives (1)-(486)”).

In certain embodiments, derivatives of indirubin may include any of the derivatives described in US20140275168A1, US20160243077A1, US20070276025A1, U.S. Pat. Nos. 9,051,306B2, 8,859,783B2, 8,829,203B2, 8,552,053B2, 7,572,923B2, EP2518139A1, or WO2014053580A1 (all incorporated by reference).

### Diseases Treatable by Indirubin and Derivatives Thereof

The subject pharmaceutical formulation comprising indirubin and derivatives thereof (or in short, “indirubin and derivatives thereof”) may be used to treat a variety of diseases. These diseases include but are not limited to cancer including chronic myelogenous leukemia (CML) and glioblastomas, neurodegenerative disorders including Alzheimer's disease, inflammatory diseases including psoriasis, or any disease associated with GSK-3 (such as Type II diabetes (Diabetes mellitus type 2), Alzheimer's Disease, inflammation, cancer (e.g., glioma and pancreatic cancer), and bipolar disorder.

In certain embodiments, the cancer is glioma, glioblastoma, medullablastoma, pancreatic cancer, leukemia such as B-cell acute lymphoblastic leukemia, B-cell chronic lymphocytic leukemia, AML (acute myelogenous leukemia) and CML (chronic myelogenous leukemia), non-Hodgkin's lymphoma, Burkett's lymphoma, follicular like lymphoma, diffuse large B-cell lymphoma, marginal zone cell lymphoma, mantle cell lymphoma, colorectal cancer, retinoblastoma, squamous cell carcinoma of the head and neck (HNSCC), prostate cancer, breast cancer, endometrial cancer, lung cancer, bladder cancer, testicular cancer, ovarian cancer (such as taxol-resistant ovarian cancer), thyroid cancer, bone cancer, stomach cancer, hepatic cancer, renal cancer, chondrocytoma, small cell lung carcinoma, large-cell lung carcinoma, non-small cell lung carcinoma, lung epidermoid and adenocarcinoma, cervical carcinomas, osteosarcoma, and melanoma. In certain embodiments, the cancer is B cell proliferative disorder, such as mantle cell lymphoma, chronic lymphocytic leukemia (CLL), small lymphocytic lymphoma (SLL), diffuse large B-cell lymphoma (DLBCL), activated B-cell diffuse large B-cell lymphoma (ABC-DLBCL), germinal center diffuse large B-cell lymphoma (GCB DLBCL), double-hit (DH) DLBCL, primary mediastinal B-cell lymphoma (PMBL), Burkett's lymphoma, follicular lymphoma, immunoblastic large cell lymphoma, precursor B-lymphoblastic lymphoma, precursor B-cell acute lymphoblastic leukemia, hairy cell leukemia B cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis. In certain embodiments, the B cell proliferative disorder is an ibrutinib-resistant B cell proliferative disorder, or an ibrutinib-resistant mantle cell lymphoma.

In certain embodiments, the cancer is one in which FGFR1 is upregulated and/or in which FGFR1 mediated-signaling is upregulated.

In certain embodiments, indirubin and derivatives thereof may be used to treat an inflammatory disease.

In certain embodiments, the inflammatory disease is an inflammatory dermatological condition, such as psoriasis.

In certain embodiments, indirubin and derivatives thereof may be used to treat an inflammatory-related disease or disorder such as diabetes, nephropathy, obesity, hearing loss, fibrosis related disease, arthritis, allergy, allergic rhinitis, acute respiratory distress syndrome, asthma, bronchitis, inflammatory bowel disease, an autoimmune disease, hepatitis, atopic dermatitis, pemphigus, glomerulonephritis, atherosclerosis, sarcoidosis, ankylosing spondylitis, Wegner's syndrome, Goodpasture's syndrome, giant cell arteritis, polyarteritis nodosa, idiopathic pulmonary fibrosis, acute lung injury, chronic obstructive pulmonary disease, post-influenza pneumonia, SARS, tuberculosis, malaria, sepsis, cerebral malaria, Chagas disease, schistosomiasis, bacterial and viral meningitis, cystic fibrosis, multiple sclerosis, Alzheimer's disease, encephalomyelitis, sickle cell anemia, pancreatitis, transplantation, systemic lupus erythematosis, thyroiditis, and radiation pneumonitis, lymphocytosis syndrome, or lymphocytic interstitial pneumonitis.

In certain embodiments, the diabetes is Type II diabetes, Type I diabetes, diabetes insipidus, diabetes mellitus, maturity-onset diabetes, juvenile diabetes, insulin-dependent diabetes, non-insulin dependent diabetes, malnutrition-related diabetes, autoimmune diabetes, ketosis-prone diabetes or ketosis-resistant diabetes.

In certain embodiments, the nephrophaty is glomerulonephritis, acute kidney failure or chronic kidney failure.

In certain embodiments, the obesity is hereditary obesity, dietary obesity, hormone related obesity or obesity related to the administration of medication.

In certain embodiments, the hearing loss results from otitis extema or acute otitis media.

In certain embodiments, the fibrosis related disease is pulmonary interstitial fibrosis, renal fibrosis, cystic fibrosis, liver fibrosis, wound-healing or burn-healing.

In certain embodiments, the arthritis is rheumatoid arthritis, rheumatoid spondylitis, psoriatic arthritis, osteoarthritis or gout.

In certain embodiments, the irritable bowel disease is irritable bowel syndrome, mucous colitis, ulcerative colitis, Crohn's disease, gastritis, esophagitis, pancreatitis or peritonitis.

In certain embodiments, the autoimmune disease is scleroderma, systemic lupus erythematosus, myasthenia gravis, transplant rejection, endotoxin shock, sepsis, psoriasis, eczema, dermatitis or multiple sclerosis.

In certain embodiments, the hepatitis is viral chronic hepatitis.

In certain embodiments, indirubin and derivatives thereof may be used to treat an ocular disease characterized by inflammation of the eye or adnexa of the eye in a patient suffering therefrom, such as dry eye disease or Sjogren's disease.

In certain embodiments, indirubin and derivatives thereof may be used to treat skin disorder, including skin inflammation. In certain embodiments, the skin disorder is selected from the group consisting of atopic dermatitis, acne or psoriasis, more preferably psoriasis. In certain embodiments, the skin disorder is an inflammatory skin condition, onychomycosis, skin cancer, abnormal keratinization induced diseases, skin aging, pustular dermatosis, atopic dermatitis (AD), eczema, superinfected skin, abnormal keratinization (such as acne, ichtyosis and palmoplantar keratoderma).

In certain embodiments, the psoriasis is chronic plaque psoriasis, guttate psoriasis, erythrodermic psoriasis, pustular psoriasis, psoriatic skin lesions, psoriatic nail lesions, and the combinations thereof.

In certain embodiments, indirubin and derivatives thereof may be used to treat a neurological disorder. In certain embodiments, indirubin and derivatives thereof may be used to regenerate nerve in a neurological disorder.

In certain embodiments, the neurological disorder is Parkinson's disease, Huntington's disease, Alzheimer's disease, Down's disease, cerebrovascular disorder, cerebral stroke, ischemias of the brain and neurotraumas, spinal cord injury, Huntington's chorea, multiple sclerosis, amyotrophic lateral sclerosis, epilepsy, anxiety disorder, schizophrenia, dopamine dysregulation, depression and manic depressive psychosis.

In certain embodiments, the neurological disorder is age associated memory impairment (AAMI), mild cognitive impairment (MCI), Alzheimer's disease (AD), cerebrovascular dementia (CVD) and related retrogenic degenerative neurological conditions.

In certain embodiments, indirubin and derivatives thereof may be used to inhibit the replication of a pathogenic agent, such as a virus, a bacterium, a fungus, a yeast or a parasite.

In certain embodiments, the virus is a herpesvirus (such as herpes simplex virus type 1 (HSV-1), herpes simplex virus type 2 (HSV-2), cytomegalovirus, varicella zoster virus (VZV), bovine herpesvirus type 1 (BHV-1), equine herpesvirus type 1 (EHV-1), pseudorabiesvirus (PRV), Epstein Barr virus, human herpesvirus type 6, human herpesvirus type 7 and human herpesvirus type 8), a hepatitis B virus, a hepatitis C virus, a human papilloma virus, human immunodeficiency virus (HIV), flavivirus, or human T-cell leukemia virus (HTLV).

In certain embodiments, indirubin and derivatives thereof may be used to treat HIV infection, or HIV-1 associated dementia (HAD) such as minor cognitive minor motor disease (MCMD).

In certain embodiments, indirubin and derivatives thereof may be used to treat Gram-positive bacterial infection associated with increased activity of a bacterial serine/threonine protein kinase.

In certain embodiments, indirubin and derivatives thereof may be used to treat infection by Staphylococcus aureus, including methicillin-resistant Staphylococcus aureus (MRSA).

In certain embodiments, indirubin and derivatives thereof may be used to treat candidiasis, such as candidiasis is caused by Candida albicans infection.

In certain embodiments, indirubin and derivatives thereof may be used to treat an injury or disease of decreased cardiac function, such as myocardial infarction and myocardial damage from myocardial infarction; atherosclerosis; coronary artery disease; obstructive vascular disease; dilated cardiomyopathy; heart failure; myocardial necrosis; valvular heart disease; non-compaction of the ventricular myocardium; and hypertrophic cardiomyopathy.

In certain embodiments, indirubin and derivatives thereof may be used to treat a cardiovascular disease such as stenosis, arteriosclerosis and restenosis.

In certain embodiments, indirubin and derivatives thereof may be used to induce immune tolerance in a patient or subject in need thereof. In certain embodiments, the patient has an autoimmune disease or an immune inflammatory disease. In certain embodiments, the immune inflammatory disease is systemic lupus erythematosis (SLE), diabetes mellitus (type I), asthma, arthritis, pernicious anemia, or multiple sclerosis. In certain embodiments, the autoimmune disease or said immune inflammatory disease is an autoimmune blood disease; an autoimmune disease of the musculature; an autoimmune disease of the ear; an autoimmune eye disease, an autoimmune disease of the kidney; an autoimmune skin disease; a cardiovascular autoimmune disease; an endocrine autoimmune disease; an autoimmune gastroenteric disease; an autoimmune nervous disease; and a systemic autoimmune disease. In certain embodiments, the autoimmune disease is pernicious anemia, autoimmune hemolytic anemia, aplastic anemia, idiopathic thrombocytopenic purpura, ankylosing spondylitis, polymyositis, dermatomyositis, autoimmune hearing loss, Meniere's syndrome, Mooren's disease, Reiter's syndrome, Vogt-Koyanagi-Harada disease, glomerulonephritis, IgA nephropathy; diabetes mellitus (type I), pemphigus, pemphigus vulgaris, pemphigus foliaceus, pemphigus erythematosus, bullous pemphigoid, vitiligo, epidermolysis bullosa acquisita, alopecia areata; autoimmune myocarditis, vasculitis, Churg-Strauss syndrome, giant cells arteritis, Kawasaki's disease, polyarteritis nodosa, Takayasu's arteritis and Wegener's granulomatosis, Addison's disease, autoimmune hypoparathyroidism, autoimmune hypophysitis, autoimmune oophoritis, autoimmune orchitis, Grave's Disease, Hashimoto's thyroiditis, polyglandular autoimmune syndrome type 1 (PAS-1) polyglandular autoimmune syndrome type 2 (PAS-2), and polyglandular autoimmune syndrome type 3 (PAS-3), including autoimmune hepatitis, primary biliary cirrhosis, inflammatory bowel disease, celiac disease, Crohn's disease, multiple sclerosis, myasthenia gravis, Guillain-Barre syndrome and chronic inflammatory demyelinating neuropathy, including systemic lupus erythematosus, antiphospholid syndrome, autoimmune lymphoproliferative disease, autoimmune polyendocrinopathy, Behcet's disease, Goodpasture's disease, rheumatoid arthritis, osteoarthritis, septic arthritis, sarcoidosis, scleroderma, Sjogren's syndrome, an autoimmune disease of the musculature, an autoimmune disease of the ear, an autoimmune eye disease, an autoimmune disease of the kidney, an autoimmune skin disease, a cardiovascular autoimmune disease, an endocrine autoimmune disease, an autoimmune gastroenteric disease, an autoimmune nervous disease, a systemic autoimmune disease, systemic lupus erythematosus, diabetes mellitus type I, arthritis, or multiple sclerosis.

In certain embodiments, indirubin and derivatives thereof may be used to treat or prevent longitudinal bone growth disorders. In certain embodiments, the longitudinal bone growth disorder is short stature, microplasia, dwarfism, or precocious puberty.

In certain embodiments, indirubin and derivatives thereof may be used to treat a c-Met-induced or angiogenesis factor-induced disease, such as cancer, gestational diabetes, diabetic retinopathy, or macular degeneration.

In certain embodiments, indirubin and derivatives thereof may be used to treat Duchenne Muscular Dystrophy (DMD), or a non-human model of DMD.

In certain embodiments, indirubin and derivatives thereof may be used to treat sepsis, arteriosclerosis, acute coronary syndrome, stroke, emphysema, acute respiratory distress syndrome, osteoporosis, hypertension, obesity, diabetes, arthritis, or a cerebral disease.

In certain embodiments, indirubin and derivatives thereof may be used to treat mouth ulcer, oral cancer, esophagitis, esophageal cancer, gastritis, duodenal ulcer, stomach cancer, inflammatory bowel disease, irritable bowel syndrome, colorectal cancer, cholangitis, cholecystitis, pancreatitis, cholangiocarcinoma, and pancreatic cancer.

In certain embodiments, indirubin and derivatives thereof may be used to treat Castiemarf s Disease, lupus, multiple sclerosis, scleroderma pigmentosa, Autoimmune Lymphoproliferative Syndrome (ALPS), myesthenia gravis, diabetes, asthma, rheumatoid arthritis, vitiligo, diGeorge's syndrome, Grave's disease, pemphigus vulgaris, Crohn's disease, inflammatory bowel disease, colitis, orchitis, uveitis, Post-Transplant Lymphoproliferative Disease (PTLD), or Autoimmune disease-associated lymphadenopathy (ADALA).

### Pharmaceutical Excipients

Pharmaceutical compositions according to the disclosure may also comprise pharmaceutical excipients. These are one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC®).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

### Methods of Using Nanoparticulate Indirubin Formulations Described Herein

1. Applications of the Nanoparticulate Compositions

The nanoparticulate indirubin compositions described herein may be used to treat any of the diseases and conditions described in the section above, entitled “Diseases Treatable by Indirubin and Derivatives Thereof.”

In certain embodiments, the nanoparticulate indirubin compositions described herein may be used to treat cancer, including any cancer described in the section above entitled “Diseases Treatable by Indirubin and Derivatives Thereof.” For example, the nanoparticulate indirubin compositions described herein may also be used to treat leukemia, especially chronic myelogenous leukemia (CML) and glioblastomas.

The nanoparticulate indirubin compositions described herein may also be used to treat inflammatory diseases including psoriasis.

The nanoparticulate indirubin compositions described herein may further be used to treat neurodegenerative disorders including Alzheimer's disease.

The nanoparticulate indirubin compositions described herein may also be used to treat any other disease associated with GSK-3.

Glycogen synthase kinase 3 (GSK-3) is a serine/threonine protein kinase that mediates the addition of phosphate molecules onto serine and threonine amino acid residues. GSK-3 has been identified as a kinase for over forty different proteins in a variety of different pathways. In mammals, GSK-3 is encoded by two known genes, GSK-3 alpha (GSK3A) and GSK-3 beta (GSK3B). Due to its involvement in a great number of signaling pathways, GSK-3 has been associated with a host of high-profile diseases, including Type II diabetes (Diabetes mellitus type 2), Alzheimer's Disease, inflammation, cancer (e.g., glioma and pancreatic cancer), and bipolar disorder.

2. Dosage Forms

The nanoparticulate indirubin compositions described herein can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, gels, creams, ointments or drops), or as a buccal or nasal spray. As used herein, the term “subject” is used to mean an animal, preferably a mammal, including a human or non-human. The terms patient and subject may be used interchangeably. The nanoparticulate indirubin compositions described herein can also be administered to the central nervous system, e.g., to the brain or spinal cord. In certain embodiments, the nanoparticulate indirubin compositions described herein are administered to the brain. According to certain embodiments, the nanoparticulate indirubin compositions described herein are administered with an agent that enhances the permeability of the blood brain barrier (BBB) to nanoparticulate indirubin compositions.

Moreover, the nanoparticulate indirubin compositions described herein can be formulated into any suitable dosage form, including but not limited to liquid dispersions, gels, aerosols, ointments, creams, controlled release formulations, fast melt formulations, lyophilized formulations, tablets, capsules, delayed release formulations, extended release formulations, pulsatile release formulations, and mixed immediate release and controlled release formulations.

Nanoparticulate indirubin compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate indirubin compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffeting agents.

Liquid nanoparticulate indirubin dosage forms for oral administration include pharmaceutically acceptable emulsions, solutions, suspensions, syrups, and elixirs. In addition to indirubin, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

The following examples are given for illustrative purposes. It should be understood, however, that the nanoparticulate indirubin composition described herein are not to be limited to the specific conditions or details described in these examples. Throughout the specification, any and all references to a publicly available document, including a U.S. patent, are specifically incorporated by reference.

In the examples that follow, the value for D50 is the particle size below which 50% of the indirubin particles fall. Similarly, D90 is the particle size below which 90% of the indirubin particles fall.

The formulations in the examples that follow were also investigated using a light microscope. Here, “stable” nanoparticulate dispersions (uniform Brownian motion) were readily distinguishable from “aggregated” dispersions (relatively large, nonuniform particles without motion). Stable, as known in the art and used herein, means the particles don't substantially aggregate or ripen (increase in fundamental particle size).

### EXAMPLES

### Example 1

### Single Emulsion

Dissolve 10 mg of 6-bromoindirubin-3′-oxime (6-BIA) and 150 mg of polylactide (PLA) in 3 ml of ethyl acetate to form a PLA-indirubin solution. This solution is mixed with 10 ml of 5% aqueous solution of polyvinyl alcohol in a glass vial and ultrasonicated with a probe sonicator at 60% of powder output for 45 seconds. The resulting emulsion is stirred magnetically for 2 hours to allow ethyl acetate to evaporate.

The PLGA-encapsulated 6-BIA nanoparticles obtained are found to have an average particle size of 220 nm.

### Example 2

### Precipitation Method

Dissolve 10.0 mg 6-BIA and 20.0 mg of poly(ethylene glycol-co-polylactide), AK31 of PolyScitech in 2 mL acetone by vortex and sonication; Prepare 20 mL of aqueous solution in a 30-mL beaker containing 0.5% HPMC E3+2% PVA (80% hydrolyzed), while stirring at 600 rpm, add AK31/6-BIA solution to the aqueous solution using a 1 mL syringe with 27G needle, followed by stirring for 30-60 min and allowing acetone to evaporate.

After washing and filtration, the encapsulated 6-BIA particle size was measured and found to be 86 nm.

### Example 3

### Precipitation Method

10 mg of indirubin and 150 mg of PLGA are dissolved in 10 ml dimethyl sulfoxide (DMSO). The indirubin-PLGA solution is then added dropwise to a beaker containing 200 ml of 5% by weight polyvinyl alcohol solution while stirring. The resulting indirubin nanoparticles are purified by tangential flow filtration.

Particle size analysis is performed with a Malvern particle size analyzer (Worcestershire, UK). The average encapsulated indirubin particle size is found to be 225.0 nm, and indirubin loading is found to be 2%.

### Example 4

### Precipitation Method

Dissolve 10.0 mg 6-BIA and 20.0 mg of poly(ethylene glycol-co-polylactide), AK31 of PolyScitech in 2 mL acetone by vortex and sonication; Prepare 20 mL of aqueous solution in a 30-mL beaker containing 0.5% HPMC E3+2% PVA (80% hydrolyzed), while stirring at 600 rpm, add AK31/6-BIA solution to the aqueous solution using a 1 mL syringe with 27G needle, followed by stirring for 30-60 min and allowing acetone to evaporate.

After washing and filtration, the encapsulated 6-BIA particle size was measured and found to be 67.5 nm.

### Example 5

### Solubility Measurement of 6-BIA

As a control, the dissolution of 6-BIA without polymer was tested. 1.10 mg of 6-BIA was added to 110 mL of 2% Tween 20 in PBS. After brief stirring 1 mL of the resulting suspension was immediately collected to an Eppendorf tube. The Eppendorf tube was centrifuged at 14,000 rpm for 15 min. 800 μL of the supernatant was collected and measured for 6-BIA concentration by HPLC (292 nm) and was found to be 2.20 μg/ml.

### Example 6

### Solubility Measurement of 6-BIA Nanoparticles

Encapsulated 6-BIA nanoparticles obtained in Example 4 were re-suspended in 2% Tween 20 in PBS to form a nanoparticle suspension containing approximately 1.32 mg/ml of encapsulated 6-BIA in the nanoparticles. After brief stirring, 1 mL of the resulting suspension was immediately collected to an Eppendorf tube. The Eppendorf tube was centrifuged at 14,000 rpm for 15 min. 800 μL of the supernatant was collected and measured for 6-BIA concentration by HPLC (292 nm), which was found to be 5.72 μg/ml.

Thus, the encapsulated 6-BIA nanoparticles demonstrated higher instant solubility than 6-BIA itself (5.72 μg/ml vs. 2.20 μg/ml in comparative Example 5).

### Example 7

### Dissolution Measurement of 6-BIA After 30 Minutes

As a control, dissolution of 6-BIA without polymer was first tested. 1.10 mg of 6-BIA was added to 110 mL of 2% Tween 20 in PBS. After stirring for 30 minutes, 1 mL of the resulting suspension was immediately collected to an Eppendorf tube. The Eppendorf tube was centrifuged at 14,000 rpm for 15 min. 800 μL of the supernatant was collected and measured for 6-BIA concentration by HPLC (292 nm), which was found to be 3.89 μg/ml.

### Example 8

### Dissolution Measurement of 6-BIA Nanoparticles After 30 Minutes

Encapsulated 6-BIA nanoparticles obtained in Example 4 were re-suspended in 2% Tween 20 in PBS to form a nanoparticle suspension containing approximately 1.32 mg/ml of 6-BIA in the nanoparticles. After stirring for 30 minutes, 1 mL of the resulting suspension was immediately collected to an Eppendorf tube. The Eppendorf tube was centrifuged at 14,000 rpm for 15 min. 800 μL of the supernatant was collected and measured for 6-BIA concentration by HPLC (292 nm), which was found to be 8.05 μg/ml.

Thus, the encapsulated 6-BIA nanoparticles demonstrated higher 30-minute dissolution than 6-BIA itself (8.05 μg/ml vs. 3.89 μg/ml in comparative Example 7).

1-50. (canceled)

51. A pharmaceutical formulation comprising 6-bromoindirubin-3′-oxime or 6-bromoindirubin-3′-acetoxime, and a pharmaceutically acceptable polymer, wherein the pharmaceutically acceptable polymer:
(1) encapsulates the 6-bromoindirubin-3′-oxime or the 6-bromoindirubin-3′-acetoxime to form particulates with an average particle size of about 1 nm to about 1,000 nm; and,
(2) is selected from the group consisting of: PLA, PLGA, PEG-PLGA copolymer, PEG-PLA copolymer, PEG-PGA copolymer, poly(ethylene glycol), polycaprolactone, polyanhydrides, poly(ortho esters), polycyanoacrylates, poly(hydroxyalkanoate)s, poly(sebasic acid), polyphosphazenes, polyphosphoesters, modified poly(saccharide)s, and mixtures and copolymers thereof.

52. The pharmaceutical formulation of claim 51, wherein the average particle size of the particulates is about 10 nm to about 300 nm, about 20-500 nm, about 20 nm to about 200 nm, about 50-100 nm; or about 100 nm.

53. The pharmaceutical formulation of claim 51, wherein solubility in an aqueous solution of said 6-bromoindirubin-3′-oxime or said 6-bromoindirubin-3′-acetoxime in said pharmaceutical formulation is at least about 100%, 2-fold, 3-fold, 5-fold, 10-fold, 20-fold, 50-fold, or 100-fold of that said 6-bromoindirubin-3′-oxime or said 6-bromoindirubin-3′-acetoxime in the same aqueous solution.

54. The pharmaceutical formulation of claim 51, wherein the pharmaceutically acceptable polymer is PLA, PLGA, or a copolymer of PLGA.

55. The pharmaceutical formulation of claim 54, wherein the pharmaceutically acceptable polymer is PLGA or PEG-PLGA.

56. The pharmaceutical formulation of claim 51, wherein said particulates have an incorporated color dye or fluorescent dye.

57. The pharmaceutical formulation of claim 51, comprising 6-bromoindirubin-3′-acetoxime.

58. A method of producing the pharmaceutical formulation of claim 51, wherein the method is:
(1) a single emulsion process comprising:
(a1) dissolving 6-bromoindirubin-3′-oxime or 6-bromoindirubin-3′-acetoxime along with the pharmaceutically acceptable polymer in a first solvent to form a polymer-indirubin solution;
(b1) emulsifying the polymer-indirubin solution in a second solvent to form an emulsion, wherein the first solvent is not miscible or only partially miscible with the second solvent; and
(c1) removing the first solvent to form the particulates;,
(2) a double emulsion process comprising:
(a2) dissolving 6-bromoindirubin-3′-oxime or 6-bromoindirubin-3′-acetoxime along with the pharmaceutically acceptable polymer in a first solvent to form a polymer-indirubin solution;
(b2) adding a small amount (e.g., 0.5% (v/v), 1% (v/v), 5% (v/v)) of a second solvent to the polymer-indirubin solution to form a mixture, wherein the first solvent is not miscible or only partially miscible with the second solvent;
(c2) emulsifying the mixture to form a first emulsion;
(d2) emulsifying the first emulsion in a third solvent to form a second emulsion; and,
(e2) removing the first solvent to form said particles; or,
(3) a precipitation process comprising:
(a3) dissolving 6-bromoindirubin-3′-oxime or 6-bromoindirubin-3′-acetoxime in a first solvent along with the pharmaceutically acceptable polymer;
(b3) optionally adding to the first solvent a first solution comprising a surface stabilizer to form a formulation; and,
(c3) precipitating the formulation from step (2) into a second solution containing the surface stabilizer in a second solvent, wherein the second solvent is miscible with the first solvent and is a non-solvent for both the polymer and the indirubin or the derivative thereof.

59. A method of treating a cancer in a subject in need thereof, the method comprising administering an effective amount of the pharmaceutical composition of claim 51.

60. The method of claim 59, wherein the cancer is a glioblastoma.

61. The method of claim 59, wherein the cancer is a pancreatic cancer.

62. The method of claim 59, wherein said subject is a human.

63. A method of treating a disorder associated with abnormal GSK-3 activity, in a subject in need thereof, the method comprising administering to the subject an effective amount of the pharmaceutical composition of claim 51.

64. The method of claim 63, wherein the disorder is Type II diabetes (Diabetes mellitus type 2), Alzheimer's Disease, inflammation, cancer, or bipolar disorder.

65. The method of claim 64, wherein said subject is a human.
